SEQUENCE LISTING (15 sequences)

SEQ ID NO: 1 - DNA, 384 bp, Mouse
  misc_feature (1)...(384): n = A,T,C or G
ggtggacttcggtgggacaacgtccttccagtgcaaggtgcgcagtgacgtgaagcctgtgatccagtggctgaagcgggtggagtacggctccgagggacgccacaactccaccattgatgtgggtggccagaagtttgtggtgttgcccacgggtgatgtgtggtcacggcctgatggctcctacctcaacaagctgctcatctctcgggcccgccaggatgatgctggcatgtacatctgcctaggtgcaaataccatgggctacagtttccgtagcgccttcctcactgtattaccagaccccaaacctccagggcctcctatggcttcttcatcgtcatccacaagcctgccatggcctgtggngatcggcatcccagc

SEQ ID NO: 2 - DNA, 1967 bp, Mouse
gctgcgcgcccccgcgctgatccctgtcgagcgtctacgcgcctcgcttcctttgcctggagctcggcgccgaggggggccggaccctggctctgcggccgcgacctgggtcttgcgggcctgagccctgagtggcgtccagtccagctcccagtgaccgcgcccctgcttcaggtccgaccggcgagatgacgcggagccccgcgctgctgctgctgctattgggggccctcccgtcggctgaggcggcgcgaggacccccaagaatggcagacaaagtggtcccacggcaggtggcccgcctgggccgcactgtgcggctacagtgcccagtggagggggacccaccaccgttgaccatgtggaccaaagatggccgcacaatccacagtggctggagccgcttccgtgtgctgccccagggtctgaaggtgaaggaggtggaggccgaggatgccggtgtttatgtgtgcaaggccaccaatggctttggcagcctcagcgtcaactacactctcatcatcatggatgatattagtccagggaaggagagccctgggccaggtggttcttcggggggccaggaggacccagccagccagcagtgggcacggcctcgcttcacacagccctccaagatgaggcgccgagtgattgcacggcctgtgggtagctctgtgcggctcaagtgtgtggccagtgggcacccacggccagacatcatgtggatgaaggatgaccagaccttgacgcatctagaggctagtgaacacagaaagaagaagtggacactgagcttgaagaacctgaagcctgaagacagtggcaagtacacgtgccgtgtatctaacaaggccggtgccatcaacgccacctacaaagtggatgtaatccagcggactcgttccaagcctgtgctcacagggacacaccctgtgaacacaacggtggacttcggtgggacaacgtccttccagtgcaaggtgcgcagtgacgtgaagcctgtgatccagtggctgaagcgggtggagtacggctccgagggacgccacaactccaccattgatgtgggtggccagaagtttgtggtgttgcccacgggtgatgtgtggtcacggcctgatggctcctacctcaacaagctgctcatctctcgggcccgccaggatgatgctggcatgtacatctgcctaggtgcaaataccatgggctacagtttccgtagcgccttcctcactgtattaccagaccccaaacctccagggcctcctatggcttcttcatcgtcatccacaagcctgccatggcctgtggtgatcggcatcccagctggtgctgtcttcatcctaggcactgtgctgctctggctttgccagaccaagaagaagccatgtgccccagcatctacacttcctgtgcctgggcatcgtcccccagggacatcccgagaacgcagtggtgacaaggacctgccctcattggctgtgggcatatgtgaggagcatggatccgccatggccccccagcacatcctggcctctggctcaactgctggccccaagctgtaccccaagctatacacagatgtgcacacacacacacatacacacacctgcactcacacgctctcatgtggagggcaaggttcatcaacaccagcatgtccactatcagtgctaaatacagcgaatctccaagcactgtgtcctgaggtaggcatatgggggccaaggcaacaggttgggagaattgagaacaatggaggaagagtatcttagggtgccttatggtggacactcacaaacttggccatatagatgtatgtactaccagatgaacagccagccagattcacacacgcacatgtttaaacgtgtaaacgtgtgcacaactgcacacacaacctgagaaaccttcaggaggatttggggtgtgactttgcagtgacatgtagcgatggctagttg

SEQ ID NO: 3 - DNA, 1742 bp, Mouse
gcgcggcgccccgggcccctcgccccgccgcccctcttccccgccctcgccaagcctcgccgtttatccgcgcggacagcgcgccccgcgccccagcccggccctagccgccagcgcccaggtagcgccgccccgcccaggccgggcccgggggcgcggggggcgggatgcggcgcccggggcagcgatgaccgcgtcgcgctgctcaggggcccggctctgaccccgttgcctgctgcgcgcccccgcgctgatccctgtcgagcgtctacgcgcctcgcttcctttgcctggagctcggcgccgaggggggccggaccctggctctgcggccgcgacctgggtcttgcgggcctgagccctgagtggcgtccagtccagctcccagtgaccgcgcccctgcttcaggtccgaccggcgagatgacgcggagccccgcgctgctgctgctgctattgggggccctcccgtcggctgaggcggcgcgagatgatattagtccagggaaggagagccctgggccaggtggttcttcggggggccaggaggacccagccagccagcagtgggcacggcctcgcttcacacagccctccaagatgaggcgccgagtgattgcacggcctgtgggtagctctgtgcggctcaagtgtgtggccagtgggcacccacggccagacatcatgtggatgaaggatgaccagaccttgacgcatctagaggctagtgaacacagaaagaagaagtggacactgagcttgaagaacctgaagcctgaagacagtggcaagtacacgtgccgtgtatctaacaaggccggtgccatcaacgccacctacaaagtggatgtaatccagcggactcgttccaagcctgtgctcacagggacacaccctgtgaacacaacggtggacttcggtgggacaacgtccttccagtgcaaggtgcgcagtgacgtgaagcctgtgatccagtggctgaagcgggtggagtacggctccgagggacgccacaactccaccattgatgtgggtggccagaagtttgtggtgttgcccacgggtgatgtgtggtcacggcctgatggctcctacctcaacaagctgctcatctctcgggcccgccaggatgatgctggcatgtacatctgcctaggtgcaaataccatgggctacagtttccgtagcgccttcctcactgtattaccagaccccaaacctcctccagggcctcctatggcttcttcatcgtcatccacaagcctgccatggcctgtggtgatcggcatcccagctggtgctgtcttcatcctaggcactgtgctgctctggctttgccagaccaagaagaagccatgtgccccagcatctacacttcctgtgcctgggcatcgtcccccagggacatcccgagaacgcagtggtgacaaggacctgccctcattggctgtgggcatatgtgaggagcatggatccgccatggccccccagcacatcctggcctctggctcaactgctggccccaagctgtaccccaagctatacacagatgtgcacacacacacacatacacacacctgcactcacacgctctcatgtggagggcaaggttcatcaacaccagcatgtccactatcagtgctaaatacagcgaatctccaagcactgtgtcctgaggtaggcatatgggggccaaggcaacaggttgggagaattgagaacaatggaggaagagtatcttag

SEQ ID NO: 4 - DNA, 1004 bp, Human
gcggccgcgaccccaggtccggacaggccgagatgacgccgagccccctgttgctgctcctgctgccgccgctgctgctgggggccttcccaccggccgccgccgcccgaggccccccaaagatggcggacaaggtggtcccacggcaggtggccggctgggccgcactgtgcggctgcagtgccagtggagggggacccgccgccgctgaccatgtggaccaaggatggccgcaccatccacagcggctggagccgcttccgcgtgctgccgcaggggctgaaggtgaagcaggtggagcgggaggatgccggcgtgtacgtgtgcaaggccaccaacggcttcggcagccttagcgtcaactacaccctcgtcgtgctggatgacattagcccagggaaggagagcctggggcccgacagctcctctgggggtcaagaggaccccgccagccagcagtgggcacgaccgcgcttcacacagccctccaagatgaggcgccgggtgatcgcacggcccgtgggtagctccgtgcggctcaagtgcgtggccagcgggcaccctcggcccgacatcacgtggatgaaggacgaccaggccttgacgcgcccagaggccgctgagcccaggaagaagaagtggacactgagcctgaagaacctgcggccggaggacagcggcaaatacacctgccgcgtgtcgaaccgcgcgggcgccatcaacgccacctacaaggtggatgtgatccagcggacccgttccaagcccgtgctcacaggcacgcaccccgtgaacacgacggtggacttcggggggaccacgtccttccagtgcaaggtgcgcagcgacgtgaagccggtgatccagtggctgaagcgcgtggagtacggcgccgagggccgccacaactccaccatcgatgtgggcggccagaagtttgtggtgctgcccacgggtgacgtgtggtcgcggcccgacggctcctacctcaataagccgctccc

SEQ ID NO: 5 - protein, 126 aa, Mouse
  VARIANT (1)...(126): Xaa = Any Amino Acid
VDFGGTTSFQCKVRSDVKPVIQWLKRVEYGSEGRHNSTIDVGGQKFVVLPTGDVWSRPDGSYLNKLLISRARQDDAGMYICLGANTMGYSFRSAFLTVLPDPKPPGPPMASSSSSTSLPWPVXGIP

SEQ ID NO: 6 - protein, 529 aa, Mouse
MTRSPALLLLLLGALPSAEAARGPPRMADKVVPRQVARLGRTVRLQCPVEGDPPPLTMWTKDGRTIHSGWSRFRVLPQGLKVKEVEAEDAGVYVCKATNGFGSLSVNYTLIIMDDISPGKESPGPGGSSGGQEDPASQQWARPRFTQPSKMRRRVIARPVGSSVRLKCVASGHPRPDIMWMKDDQTLTHLEASEHRKKKWTLSLKNLKPEDSGKYTCRVSNKAGAINATYKVDVIQRTRSKPVLTGTHPVNTTVDFGGTTSFQCKVRSDVKPVIQWLKRVEYGSEGRHNSTIDVGGQKFVVLPTGDVWSRPDGSYLNKLLISRARQDDAGMYICLGANTMGYSFRSAFLTVLPDPKPPGPPMASSSSSTSLPWPVVIGIPAGAVFILGTVLLWLCQTKKKPCAPASTLPVPGHRPPGTSRERSGDKDLPSLAVGICEEHGSAMAPQHILASGSTAGPKLYPKLYTDVHTHTHTHTCTHTLSCGGQGSSTPACPLSVLNTANLQALCPEVGIWGPRQQVGRIENNGGRVS

SEQ ID NO: 7 - protein, 439 aa, Mouse
MTRSPALLLLLLGALPSAEAARDDISPGKESPGPGGSSGGQEDPASQQWARPRFTQPSKMRRRVIARPVGSSVRLKCVASGHPRPDIMWMKDDQTLTHLEASEHRKKKWTLSLKNLKPEDSGKYTCRVSNKAGAINATYKVDVIQRTRSKPVLTGTHPVNTTVDFGGTTSFQCKVRSDVKPVIQWLKRVEYGSEGRHNSTIDVGGQKFVVLPTGDVWSRPDGSYLNKLLISRARQDDAGMYICLGANTMGYSFRSAFLTVLPDPKPPPGPPMASSSSSTSLPWPVVIGIPAGAVFILGTVLLWLCQTKKKPCAPASTLPVPGHRPPGTSRERSGDKDLPSLAVGICEEHGSAMAPQHILASGSTAGPKLYPKLYTDVHTHTHTHTCTHTLSCGGQGSSTPACPLSVLNTANLQALCPEVGIWGPRQQVGRIENNGGRVS

SEQ ID NO: 8 - protein, 322 aa, Human
RRAPCCCSCCRRCCWGPSHRPPPPEAPQRWRTRWSHGRWPAGPHCAAAVPVEGDPPPLTMWTKDGRTIHSGWSRFRVLPQGLKVKQVEREDAGVYVCKATNGFGSLSVNYTLVVLDDISPGKESLGPDSSSGGQEDPASQQWARPRFTQPSKMRRRVIARPVGSSVRLKCVASGHPRPDITWMKDDQALTRPEAAEPRKKKWTLSLKNLRPEDSGKYTCRVSNRAGAINATYKVDVIQRTRSKPVLTGTHPVNTTVDFGGTTSFQCKVRSDVKPVIQWLKRVEYGAEGRHNSTIDVGGQKFVVLPTGDVWSRPDGSYLNKPL

SEQ ID NO: 9 - DNA, 1423 bp, Mouse
tctgtgcggctcaagtgtgtggccagtgggcacccacggccagacatcatgtggatgaaggatgaccagaccttgacgcatctagaggctagtgaacacagaaagaagaagtggacactgagcttgaagaacctgaagcctgaagacagtggcaagtacacgtgccgtgtatctaacaaggccggtgccatcaacgccacctacaaagtggatgtaatccgtgagtggtgggtctgtggtaggacaggggcccgtggtgcctaaaactgtgctgacatgtttgtttttccttggcttagagcggactcgttccaagcctgtgctcacagggacacaccctgtgaacacaacggtggacttcggtgggacaacgtccttccagtgcaaggtgcgcagtgacgtgaagcctgtgatccagtggctgaagcgggtggagtacggctccgagggacgccacaactccaccattgatgtgggtggccagaagtttgtggtgttgcccacgggtgatgtgtggtcacggcctgatggctcctacctcaacaagctgctcatctctcgggcccgccaggatgatgctggcatgtacatctgcctaggtgcaaataccatgggctacagtttccgtagcgccttcctcactgtattaccaggtgtgtgtgtgggctgcccaccccatgtttactctcagtctctaccattggtctgggctgtcctggggttccccaatgtccacttagcaagtggggcctccctatccttttcccttcgttgtgggttatccttgcctcatagggagttcaggggtgctgcccatatagttcacatttgggctggttgccccattaatatagggacattctgtcccctactcttcttcttaatctctcttgcagaccccaaacctccagggcctcctatggcttcttcatcgtcatccacaagcctgccatggcctgtggtgatcggcatcccagctggtgctgtcttcatcctaggcactgtgctgctctggctttgccagaccaagaagaagccatgtgccccagcatctacacttcctgtgcctgggcatcgtcccccagggacatcccgagaacgcagtggtgacaaggacctgccctcattggctgtgggcatatgtgaggagcatggatccgccatggccccccagcacatcctggcctctggctcaactgctggccccaagctgtaccccaagctatacacagatgtgcacacacacacacatacacacacctgcactcacacgctctcatgtggagggcaaggttcatcaacaccagcatgtccactatcagtgctaaatacagcgaatctccaagcactgtgtcctgaggtaggcatatgggggccaaggcaacaggttgggagaattgagaacaatggaggaagagtatct

SEQ ID NO: 10 - DNA, 35 bp, Artificial sequence
  Made in a lab from synthetic material
cgggatccaggccatggcaggcttgtggatgacga

SEQ ID NO: 11 - DNA, 37 bp, Artificial sequence
  Made in a lab from synthetic material
ccgctcgagtagatactcttcctccattgttctcatt

SEQ ID NO: 12 - DNA, 18 bp, Artificial sequence
  Made in a lab from synthetic material
ctgtgcggctcaagtgtg

SEQ ID NO: 13 - protein, 373 aa, Mouse
MTRSPALLLLLLGALPSAEAARGPPRMADKVVPRQVARLGRTVRLQCPVEGDPPPLTMWTKDGRTIHSGWSRFRVLPQGLKVKEVEAEDAGVYVCKATNGFGSLSVNYTLIIMDDISPGKESPGPGGSSGGQEDPASQQWARPRFTQPSKMRRRVIARPVGSSVRLKCVASGHPRPDIMWMKDDQTLTHLEASEHRKKKWTLSLKNLKPEDSGKYTCRVSNKAGAINATYKVDVIQRTRSKPVLTGTHPVNTTVDFGGTTSFQCKVRSDVKPVIQWLKRVEYGSEGRHNSTIDVGGQKFVVLPTGDVWSRPDGSYLNKLLISRARQDDAGMYICLGANTMGYSFRSAFLTVLPDPKPPGPPMASSSSSTSLPW

SEQ ID NO: 14 - protein, 135 aa, Mouse
CQTKKKPCAPASTLPVPGHRPPGTSRERSGDKDLPSLAVGICEEHGSAMAPQHILASGSTAGPKLYPKLYTDVHTHTHTHTCTHTLSCGGQGSSTPACPLSVLNTANLQALCPEVGIWGPRQQVGRIENNGGRVS

SEQ ID NO: 15 - protein, 37 aa, Mouse
RVEYGSEGRHNSTIDVGGQKFVVLPTGDVWSRPDGSY

(19) United States
(12) Patent Application Publication
(10) Pub. No.: US 2003/0143676 A1
(43) Pub. Date: Jul. 31, 2003

(54) Fibroblast growth factor receptors and methods for their use

(75) Inventors: Lorna Strachan, Auckland (NZ); Matthew Sleeman, Weston Colville (GB); Nevin Abernethy, Auckland (NZ); Rene Onrust, Seattle, WA (US); Krishanand Kumble, Los Altos, CA (US); James Greg Murison, Auckland (NZ)

Correspondence Address: SPECKMAN LAW GROUP, 1501 WESTERN AVE, SUITE 100, SEATTLE, WA, 98101, US

(73) Assignee: Genesis Research and Development Corporation Limited, Auckland (NZ)

(21) Appl. No.: 10/157,444

(22) Filed: May 28, 2002

Related U.S. Application Data
(63) Continuation-in-part of application No. 09/823,038, filed on Mar. 28, 2001.
Application No. 09/823,038 is a continuation-in-part of application No. 09/383,586, filed on Aug. 26, 1999, now Pat. No. 6,242,419.
Application No. 09/383,586 is a continuation-in-part of application No. 09/276,268, filed on Mar. 25, 1999, now abandoned.
(60) Provisional application No. 60/221,216, filed on Jul. 25, 2000.

(30) Foreign Application Priority Data
Feb. 18, 2000 (WO) PCT/NZ00/00015

(51) Int. Cl. C12P 21/02; C12N 5/06; C07K 14/705; C07H 21/04

(52) U.S. Cl. 435/69.1; 435/320.1; 435/325; 530/350; 536/23.5

(57) ABSTRACT

Isolated fibroblast growth factor receptor polypeptides and polynucleotides encoding such polypeptides are provided, together with expression vectors and host cells comprising such isolated polynucleotides. Methods for the use of such polypeptides and polynucleotides are also provided.

### CROSS-REFERENCE TO RELATED APPLICATIONS

This application is a continuation-in-part of U.S. patent application Ser. No. 09/823,038, filed Mar. 28, 2001, which is a continuation-in-part of U.S. patent application Ser. No. 09/383,586, filed Aug. 26, 1999, now U.S. Pat. No. 6,424,419; which is a continuation-in-part of U.S. patent application Ser. No. 09/276,268, filed Mar. 25, 1999, now abandoned; and claims priority to International Patent Application No. PCT/NZ00/00015, filed Feb. 18, 2000; and to U.S. Provisional Patent Application No. 60/221,216, filed Jul. 25, 2000.

### TECHNICAL FIELD OF THE INVENTION

This invention relates to polynucleotides and polypeptides derived from lymph node stromal cells from flaky skin (fsn −/−) mice, human homologues of such polynucleotides and polypeptides, and their use in therapeutic methods.

### BACKGROUND OF THE INVENTION

Lymph vessels and nodes are important components of the body's immune system. Lymph nodes are small lymphatic organs that are located in the path of lymph vessels. Large molecules and cells, including foreign substances, enter into the lymphatic vessels and, in circulating through these vessels, pass through the lymph nodes. Here, any foreign substances are concentrated and exposed to lymphocytes. This triggers a cascade of events that constitute an immune response, protecting the body from infection and from cancer.

Lymph nodes are surrounded by a dense connective tissue network that forms a supporting capsule. This network extends into the body of the lymph node, forming an additional framework of support. Throughout the remainder of the organ, a fine meshwork can be identified that comprises reticular fibres and the reticular cells that produce and surround the fibres. These features provide a support for the main functional cells of the lymphatic system, which are T- and B-lymphocytes. Additional cell types found in lymph nodes include macrophages, follicular dendritic cells, and endothelial cells that line the blood vessels servicing the node.

The cells within lymph nodes communicate with each other in order to defend the body against foreign substances. When a foreign substance, or antigen, is present, it is detected by macrophages and follicular dendritic cells that take up and process the antigen, and display parts of it on their cell surface. These cell surface antigens are then presented to T- and B-lymphocytes, causing them to proliferate and differentiate into activated T-lymphocytes and plasma cells, respectively. These cells are released into the circulation in order to seek out and destroy antigen. Some T- and B-lymphocytes will also differentiate into memory cells. Should these cells come across the same antigen at a later date, the immune response will be more rapid.

Once activated T- and B-lymphocytes are released into the circulation, they can perform a variety of functions that lead to the eventual destruction of antigen. Activated T-lymphocytes can differentiate into cytotoxic lymphocytes (also known as killer T-cells) which recognise other cells that have foreign antigens on their surface and kill the cell by causing them to lyse. Activated T-lymphocytes can also differentiate into helper T-cells which will then secrete proteins in order to stimulate B-lymphocytes, and other T-lymphocytes, to respond to antigens. In addition, activated T-lymphocytes can differentiate into suppressor T-cells which secrete factors that suppress the activity of B-lymphocytes. Activated B-lymphocytes differentiate into plasma cells, which synthesise and secrete antibodies that bind to foreign antigens. The antibody-antigen complex is then detected and destroyed by macrophages, or by a group of blood constituents known as complement.

Lymph nodes can be dissociated and the resulting cells grown in culture. Cells that adhere to the tissue culture dishes can be maintained for some length of time and are known as stromal cells. The cultured cells are a heterogeneous population and can be made up of most cells residing within lymph nodes, such as reticular cells, follicular dendritic cells, macrophages and endothelial cells. It is well known that bone marrow stromal cells play a critical role in homing, growth and differentiation of hematopoietic progenitor cells. Proteins produced by stromal cells are necessary for the maintenance of plasma cells in vitro. Furthermore, stromal cells are known to secrete factors and present membrane-bound receptors that are necessary for the survival of lymphoma cells.

An autosomal recessive mutation, designated flaky skin (fsn −/−), has been described in the inbred A/J mouse strain (The Jackson Laboratory, Bar Harbour, Me.). The mice have a skin disorder similar to psoriasis in humans. Psoriasis is a common disease affecting 2% of the population, which is characterised by a chronic inflammation associated with thickening and scaling of the skin. Histology of skin lesions shows increased proliferation of the cells in the epidermis, the uppermost layer of skin, together with the abnormal presence of inflammatory cells, including lymphocytes, in the dermis, the layer of skin below the epidermis. While the cause of the disease is unclear, psoriasis is associated with a disturbance of the immune system involving T lymphocytes. The disease occurs more frequently in family members, indicating the involvement of a genetic factor as well. Mice with the fsn gene mutation have not only a psoriatic-like skin disease but also other abnormalities involving cells of the immune and hematopoietic system. These mice have markedly increased numbers of lymphocytes associated with enlarged lymphoid organs, including the spleen and lymph nodes. In addition, their livers are enlarged, and the mice are anaemic. Genes and proteins expressed in abnormal lymph nodes of fsn −/− mice may thus influence the development or function of cells of the immune and hematopoietic system, the response of these cells in inflammatory disorders, and the responses of skin and other connective tissue cells to inflammatory signals.

There is a need in the art to identify genes encoding proteins that function to modulate all cells of the immune system. These proteins from normal or abnormal lymph nodes may be useful in modifying the immune responses to tumour cells or infectious agents such as bacteria, viruses, protozoa and worms. Such proteins may also be useful in the treatment of disorders where the immune system initiates unfavourable reactions to the body, including Type I hypersensitivity reactions (such as hay fever, eczema, allergic rhinitis and asthma), and Type II hypersensitivity reactions (such as transfusion reactions and haemolytic disease of newborns). Other unfavourable reactions are initiated during Type III reactions, which are due to immune complexes forming in infected organs during persistent infection or in the lungs following repeated inhalation of materials from moulds, plants or animals, and in Type IV reactions in diseases such as leprosy, schistosomiasis and dermatitis.

Novel proteins of the immune system may also be useful in treating autoimmune diseases where the body recognises itself as foreign. Examples of such diseases include rheumatoid arthritis, Addison's disease, ulcerative colitis, dermatomyositis and lupus. Such proteins may also be useful during tissue transplantation, where the body will often recognise the transplanted tissue as foreign and attempt to kill it, and also in bone marrow transplantation when there is a high risk of graft-versus-host disease where the transplanted cells attack their host cells, often causing death.

There thus remains a need in the art for the identification and isolation of genes encoding proteins expressed in cells of the immune system for use in the development of therapeutic agents for the treatment of disorders including those associated with the immune system.

### SUMMARY OF THE INVENTION

The present invention provides polypeptides and functional portions of polypeptides expressed in lymph node stromal cells of fsn −/− mice and human homologues of such polypeptides, together with polynucleotides encoding such polypeptides, expression vectors and host cells comprising such polynucleotides, and methods for their use.

In specific embodiments, isolated polypeptides are provided that comprise an amino acid sequence selected from the group consisting of sequences provided in SEQ ID NO: 5-8, 13-15, and variants of such sequences, as defined herein. Isolated polypeptides which comprise at least a functional portion of a polypeptide comprising an amino acid sequence selected from the group consisting of: (a) sequences provided in SEQ ID NO: 5-8, and 13-15; and (b) variants of a sequence of SEQ ID NO: 5-8, and 13-15, as defined herein, are also provided.

In other embodiments, the present invention provides isolated polynucleotides comprising a nucleotide sequence selected from the group consisting of: (a) sequences provided in SEQ ID NO: 1-4 and 9; (b) complements of sequences provided in SEQ ID NO: 1-4 and 9; (c) reverse complements of sequences provided in SEQ ID NO: 1-4; (d) reverse sequences of sequences provided in SEQ ID NO: 1-4 and 9; and (e) variants of the sequences of (a)-(d), as defined herein.

In related embodiments, the present invention provides expression vectors comprising the above polynucleotides, together with host cells transformed with such vectors.

In another aspect, the present invention provides fusion proteins comprising at least one polypeptide of the present invention.

As detailed below, the isolated polynucleotides and polypeptides of the present invention may be usefully employed in the preparation of therapeutic agents for the treatment of immunological disorders. In related embodiments, methods for modulating the growth of blood vessels, and for the treatment of disorders such as inflammatory disorders, disorders of the immune system, cancer, sarcoidal and granulomatous disorders, fibroblast growth factor-mediated disorders and viral disorders are provided. Examples of such disorders include HIV-infection; epithelial, lymphoid, myeloid, stromal and neuronal cancers; arthritis; inflammatory bowel disease; and cardiac failure.

The present invention further encompasses methods for modulating an immune response, for down-regulating the expression of osteopontin, and/or for treating disorders characterized by an elevated level of osteopontin, such as systemic lupus erythematosus, by reducing the effective amount, inactivating, and/or inhibiting the activity of an inventive polypeptide comprising a sequence of SEQ ID NO: 5-8, and 13-15 (known as FGFR5) or a variant thereof, or a polynucleotide that encodes such a polypeptide. Such methods include administering a component selected from the group consisting of: antibodies, or antigen-binding fragments thereof, that specifically bind to FGFR5; soluble FGFR5 ligands; small molecule inhibitors of FGFR5; anti-sense oligonucleotides to FGFR5; FGFR5-specific small interfering RNA molecules (siRNA or RNAi); monomeric soluble FGFR5; and engineered soluble FGFR5 molecules that bind FGFR5 ligand but do not stimulate signaling. As used herein, the term “elevated level” refers to a level that is higher than the average normal level for a specific patient population. The inventive methods may thus be employed in the treatment of disorders characterized by an abnormal or excessive level of OPN compared to levels seen in a normal healthy population.

The above-mentioned and additional features of the present invention, together with the manner of obtaining them, will be best understood by reference to the following more detailed description. All references disclosed herein are hereby incorporated by reference in their entirety as if each was incorporated individually.

### BRIEF DESCRIPTION OF THE DRAWINGS

FIG. 1 is the amino acid sequence of the murine FGF receptor muFGFβ (SEQ ID NO: 6). Several conserved domains were identified that are involved in the dimerization, ligand binding and activity of the receptor. The signal peptide and transmembrane domain are underlined, and the six cysteines conserved among the FGFR family members are in bold and underlined. Four glycosylation sites are double underlined. Three immunoglobulin domains (Ig loops) were identified (Ig loop 1: residues 40-102; Ig loop 2: residues 161-224; Ig loop 3: residues 257-341), as well as two tyrosine kinase phosphorylation sites (residues 198-201, 325-332), a cAMP- and cGMP-dependent protein kinase phosphorylation site (residues 208-215) and four prenyl group binding sites (CAAX boxes). The phosphorylation sites and CAAX boxes are boxed. A heparin binding domain was identified (residues 150-167; boxed and in bold) and this partially overlaps the CAM binding domain (residues 141-160; italics and underlined).

FIG. 2A shows the induction of genes under the control of the SRE. NIH-3T3 SRE cells were stimulated with a titration of FGF-2 in the presence of 10 μg/ml of heparin for 6 hours. Closed circles represent media alone, open squares represent titration of FGF-2. FIG. 2B shows the competition analysis of NIH-3T3 SRE cells treated with a standard dose of FGF-2 plus heparin in the presence of increasing concentrations of FGFR2Fc (closed diamonds), FGFR5γFc (closed squares), FGFR5γFc (closed triangles) and FGF-2 alone (asterisk). The mean and SD were calculated for both experiments from three separate wells and are represented as fold-induction of the reporter gene relative to control.

FIG. 3 illustrates the stimulation of growth of RAW264.10 cells by FGFR5β and FGFR5γ. This stimulation was not observed when FGF-2 and FGFR2 were used as controls. This stimulation was also not induced by the growth medium.

FIG. 4 illustrates the enhancing proliferative effect of FGFR5β and FGFR5γ on PHA-induced PBMC. The enhanced proliferation was not observed when FGFR2 or purified IgG Fc was used.

FIG. 5 shows the enhanced proliferation of anti-CD3 stimulated PBMC by FGFR5β and FGFR5γ. The enhanced proliferation was not observed when FGFR2 or purified FC was used as stimulants.

FIG. 6 demonstrates that FGFR5β and FGFR5γ, or the controls FGFR2 or IgG Fc did not stimulate proliferation of PBMC in the absence of PHA.

FIG. 7 illustrates the stimulation of PBMC adherence by FGFR5β and FGFR5γ but not by FGFR2 or purified IgG Fc.

FIG. 8 shows the stimulation of adherent PHA-stimulated PBMC by FGFR5β and FGFR5γ but not by purified IgG Fc.

FIG. 9 illustrates the stimulation of NK cell adherence by FGFR5β and FGFR5γ as measured by the presence of anti-CD56 antibodies, markers of NK cells. The filled histograms represent the adherent PBMC stained with the NK cell marker CD56 and the open histograms represent the same cells stained with the isotype-matched control antibody.

FIG. 10 shows the amino acid sequence of human FGFR5 (SEQ ID NO: 8). Several conserved domains were identified that are involved in the dimerization, ligand binding and activity of the receptor. The signal peptide is underlined, and five of the six cysteines conserved among the FGFR family members are in bold and underlined. Three immunoglobulin domains (Ig loops) were identified (Ig loop 1: residues 44-106; Ig loop 2: residues 165-228; Ig loop 3 (partial): residues 261-324), as well as a tyrosine kinase phosphorylation sites (residues 212-219), a cAMP- and cGMP-dependent protein kinase phosphorylation site (residues 202-205) and four prenyl group binding sites (CAAX boxes). The phosphorylation sites and CAAX boxes are boxed. A heparin-binding domain was identified (residues 154-171; boxed and in bold) and this partially overlaps the CAM binding domain (residues 145-164; italics and underlined).

### DETAILED DESCRIPTION OF THE INVENTION

In one aspect, the present invention provides polynucleotides isolated from lymph node stromal cells of fsn −/− mice and isolated polypeptides encoded by such polynucleotides, together with human homologues of such polynucleotides and polypeptides.

The term “polynucleotide(s),” as used herein, means a single or double-stranded polymer of deoxyribonucleotide or ribonucleotide bases and includes DNA and corresponding RNA molecules, including HnRNA and mRNA molecules, both sense and anti-sense strands, and comprehends cDNA, genomic DNA and recombinant DNA, as well as wholly or partially synthesized polynucleotides. An HnRNA molecule contains introns and corresponds to a DNA molecule in a generally one-to-one manner. An mRNA molecule corresponds to an HnRNA and DNA molecule from which the introns have been excised. A polynucleotide may consist of an entire gene, or any portion thereof. Operable anti-sense polynucleotides may comprise a fragment of the corresponding polynucleotide, and the definition of “polynucleotide” therefore includes all such operable anti-sense fragments. Anti-sense polynucleotides and techniques involving anti-sense polynucleotides are well known in the art and are described, for example, in Robinson-Benion et al., Methods in Enzymol. 254: 363-375, 1995 and Kawasaki et al., Artific. Organs 20: 836-848, 1996.

In specific embodiments, the isolated polynucleotides of the present invention comprise a polynucleotide sequence selected from the group consisting of sequences provided in SEQ ID NO: 1-4 and 9.

Complements of such isolated polynucleotides, reverse complements of such isolated polynucleotides and reverse sequences of such isolated polynucleotides are also provided, together with polynucleotides comprising at least a specified number of contiguous residues (x-mers) of any of the above-mentioned polynucleotides, extended sequences corresponding to any of the above polynucleotides, antisense sequences corresponding to any of the above polynucleotides, and variants of any of the above polynucleotides, as that term is described in this specification.

The definitions of the terms “complement”, “reverse complement” and “reverse sequence”, as used herein, are best illustrated by the following example. For the sequence 5′ AGGACC 3′, the complement, reverse complement and reverse sequence are as follows:

| | |
|---|---|
| complement | 3′ TCCTGG 5′ |
| reverse complement | 3′ GGTCCT 5′ |
| reverse sequence | 5′ CCAGGA 3′. |

Preferably, sequences that are complements of a specifically recited polynucleotide sequence are complementary over the entire length of the specific polynucleotide sequence.

Some of the polynucleotides of the present invention are “partial” sequences, in that they do not represent a full length gene encoding a full length polypeptide. Such partial sequences may be extended by analyzing and sequencing various DNA libraries using primers and/or probes and well known hybridization and/or PCR techniques. Partial sequences may be extended until an open reading frame encoding a polypeptide, a full length polynucleotide and/or gene capable of expressing a polypeptide, or another useful portion of the genome is identified. Such extended sequences, including full length polynucleotides and genes, are described as “corresponding to” a sequence identified as one of the sequences of SEQ ID NO: 1-4 and 9, or a variant thereof, or a portion of one of the sequences of SEQ ID NO: 1-4 and 9, or a variant thereof, when the extended polynucleotide comprises an identified sequence or its variant, or an identified contiguous portion (x-mer) of one of the sequences of SEQ ID NO: 1-4 and 9, or a variant thereof. Such extended polynucleotides may have a length of from about 50 to about 4,000 nucleic acids or base pairs, and preferably have a length of less than about 4,000 nucleic acids or base pairs, more preferably yet a length of less than about 3,000 nucleic acids or base pairs, more preferably yet a length of less than about 2,000 nucleic acids or base pairs. Under some circumstances, extended polynucleotides of the present invention may have a length of less than about 1,800 nucleic acids or base pairs, preferably less than about 1,600 nucleic acids or base pairs, more preferably less than about 1,400 nucleic acids or base pairs, more preferably yet less than about 1,200 nucleic acids or base pairs, and most preferably less than about 1,000 nucleic acids or base pairs.

Similarly, RNA sequences, reverse sequences, complementary sequences, antisense sequences, and the like, corresponding to the polynucleotides of the present invention, may be routinely ascertained and obtained using the cDNA sequences identified as SEQ ID NO: 1-4.

The polynucleotides identified as SEQ ID NO: 1-4 contain open reading frames (“ORFs”) or partial open reading frames encoding polypeptides or functional portions of polypeptides. Open reading frames may be identified using techniques that are well known in the art. These techniques include, for example, analysis for the location of known start and stop codons, most likely reading frame identification based on codon frequencies, etc. Open reading frames and portions of open reading frames may be identified in the polynucleotides of the present invention. Suitable tools and software for ORF analysis are well known in the art and include, for example, GeneWise, available from The Sanger Center, Wellcome Trust Genome Campus, Hinxton, Cambridge, CB10 1 SA, United Kingdom; Diogenes, available from Computational Biology Centers, University of Minnesota, Academic Health Center, UMHG Box 43 Minneapolis Minn. 55455; and GRAIL, available from the Informatics Group, Oak Ridge National Laboratories, Oak Ridge, Tennessee Tenn. Once a partial open reading frame is identified, the polynucleotide may be extended in the area of the partial open reading frame using techniques that are well known in the art until the polynucleotide for the full open reading frame is identified. Thus, open reading frames encoding polypeptides and/or functional portions of polypeptides may be identified using the polynucleotides of the present invention.

Once open reading frames are identified in the polynucleotides of the present invention, the open reading frames may be isolated and/or synthesized. Expressible genetic constructs comprising the open reading frames and suitable promoters, initiators, terminators, etc., which are well known in the art, may then be constructed. Such genetic constructs may be introduced into a host cell to express the polypeptide encoded by the open reading frame. Suitable host cells may include various prokaryotic and eukaryotic cells, including plant cells, mammalian cells, bacterial cells, algae and the like.

In another aspect, the present invention provides isolated polypeptides encoded, or partially encoded, by the above polynucleotides. The term “polypeptide”, as used herein, encompasses amino acid chains of any length including full length proteins, wherein amino acid residues are linked by covalent peptide bonds. Polypeptides of the present invention may be naturally purified products, or may be produced partially or wholly using recombinant techniques. Polypeptides may comprise a signal (or leader) sequence at the N-terminal end of the protein, which co-translationally or post-translationally directs transfer of the protein. The polypeptide may also be conjugated to a linker or other sequence for ease of synthesis, purification or identification of the polypeptide (e.g., poly-His), or to enhance binding of the polypeptide to a solid support. For example, a polypeptide may be conjugated to an immunoglobulin Fc region.

The term “polypeptide encoded by a polynucleotide” as used herein, includes polypeptides encoded by a nucleotide sequence which includes the partial isolated DNA sequences of the present invention. In specific embodiments, the inventive polypeptides comprise an amino acid sequence selected from the group consisting of sequences provided in SEQ ID NO: 5-8, 13-15, and variants of such sequences.

Polypeptides encoded by the polynucleotides of the present invention may be expressed and used in various assays to determine their biological activity. Such polypeptides may be used to raise antibodies, to isolate corresponding interacting proteins or other compounds, and to quantitatively determine levels of interacting proteins or other compounds.

All of the polynucleotides and polypeptides described herein are isolated and purified, as those terms are commonly used in the art. Preferably, the polypeptides and polynucleotides are at least about 80% pure, more preferably at least about 90% pure, and most preferably at least about 99% pure.

As used herein, the term “variant” comprehends nucleotide or amino acid sequences different from the specifically identified sequences, wherein one or more nucleotides or amino acid residues is deleted, substituted, or added. Variants may be naturally occurring allelic variants, or non-naturally occurring variants. Variant sequences (polynucleotide or polypeptide) preferably exhibit at least 75%, more preferably at least 80%, more preferably yet at least 90%, and most preferably, at least 95% or 98% identity to a sequence of the present invention. The percentage identity may be determined using well known techniques. In one embodiment, the percentage identity is determined by aligning the two sequences to be compared as described below, determining the number of identical residues in the aligned portion, dividing that number by the total number of residues in the inventive (queried) sequence, and multiplying the result by 100.

Polynucleotides and polypeptides having a specified percentage identity to a polynucleotide or polypeptide identified in one of SEQ ID NO: 1-9, 13-15 thus share a high degree of similarity in their primary structure. In addition to a specified percentage identity to a polynucleotide of the present invention, variant polynucleotides and polypeptides preferably have additional structural and/or functional features in common with a polynucleotide of the present invention. Polynucleotides having a specified degree of identity to, or capable of hybridizing to, a polynucleotide of the present invention preferably additionally have at least one of the following features: (1) they contain an open reading frame, or partial open reading frame, encoding a polypeptide, or a functional portion of a polypeptide, having substantially the same functional properties as the polypeptide, or functional portion thereof, encoded by a polynucleotide in a recited SEQ ID NO.; or (2) they contain identifiable domains in common.

Polynucleotide or polypeptide sequences may be aligned, and percentages of identical nucleotides or amino acids in a specified region may be determined against another polynucleotide or polypeptide, using computer algorithms that are publicly available. The BLASTN and FASTA algorithms, set to the default parameters described in the documentation and distributed with the algorithm, may be used for aligning and identifying the similarity of polynucleotide sequences. The alignment and similarity of polypeptide sequences may be examined using the BLASTP algorithm. BLASTX and FASTX algorithms compare nucleotide query sequences translated in all reading frames against polypeptide sequences. The FASTA and FASTX algorithms are described in Pearson and Lipman, Proc. Natl. Acad. Sci. USA 85:2444-2448, 1988; and in Pearson, Methods in Enzymol. 183:63-98, 1990. The FASTA software package is available from the University of Virginia by contacting the Assistant Provost for Research, University of Virginia, PO Box 9025, Charlottesville, Va. 22906-9025. The BLASTN software is available from the National Center for Biotechnology Information (NCBI), National Library of Medicine, Building 38A, Room 8N805, Bethesda, Md. 20894. The BLASTN algorithm Version 2.0.11 [Jan. 20, 2000] set to the default parameters described in the documentation and distributed with the algorithm, is preferred for use in the determination of polynucleotide variants according to the present invention. The use of the BLAST family of algorithms, including BLASTN, BLASTP and BLASTX, is described in the publication of Altschul et al., “Gapped BLAST and PSI-BLAST: a new generation of protein database search programs,” Nucleic Acids Res. 25:3389-3402, 1997.

The following running parameters are preferred for determination of alignments and similarities using BLASTN that contribute to the E values and percentage identity for polynucleotides: Unix running command with the following default parameters: blastall -p blastn -d embldb -e 10 -G O -E O -r 1-v 30 -b 30 -i queryseq -o results; and parameters are: -p Program Name [String]; -d Database [String]; -e Expectation value (E) [Real]; -G Cost to open a gap (zero invokes default behavior) [Integer]; -E Cost to extend a gap (zero invokes default behavior) [Integer]; -r Reward for a nucleotide match (BLASTN only) [Integer]; -v Number of one-line descriptions (V) [Integer]; -b Number of alignments to show (B) [Integer]; -i Query File [File In]; -o BLAST report Output File [File Out] Optional.

The following running parameters are preferred for determination of alignments and similarities using BLASTP that contribute to the E values and percentage identity of polypeptide sequences: blastall -p blastp d swissprotdb -e 10 -G O- E O -v 30 -b 30 i queryseq -o results; the parameters are: -p Program Name [String]; -d Database [String]; -e Expectation value (E) [Real]; -G Cost to open a gap (zero invokes default behavior) [Integer]; -E Cost to extend a gap (zero invokes default behavior) [Integer]; -v Number of one-line descriptions (v) [Integer]; -b Number of alignments to show (b) [Integer]; -I Query File [File In]; -o BLAST report Output File [File Out] Optional.

The “hits” to one or more database sequences by a queried sequence produced by BLASTN, BLASTP, FASTA, or a similar algorithm, align and identify similar portions of sequences. The hits are arranged in order of the degree of similarity and the length of sequence overlap. Hits to a database sequence generally represent an overlap over only a fraction of the sequence length of the queried sequence.

As noted above, the percentage identity of a polynucleotide or polypeptide sequence is determined by aligning polynucleotide and polypeptide sequences using appropriate algorithms, such as BLASTN or BLASTP, respectively, set to default parameters; identifying the number of identical nucleic or amino acids over the aligned portions; dividing the number of identical nucleic or amino acids by the total number of nucleic or amino acids of the polynucleotide or polypeptide of the present invention; and then multiplying by 100 to determine the percentage identity. By way of example, a queried polynucleotide having 220 nucleic acids has a hit to a polynucleotide sequence in the EMBL database having 520 nucleic acids over a stretch of 23 nucleotides in the alignment produced by the BLASTN algorithm using the default parameters. The 23-nucleotide hit includes 21 identical nucleotides, one gap and one different nucleotide. The percentage identity of the queried polynucleotide to the hit in the EMBL database is thus 21/220 times 100, or 9.5%. The percentage identity of polypeptide sequences may be determined in a similar fashion.

The BLASTN and BLASTX algorithms also produce “Expect” values for polynucleotide and polypeptide alignments. The Expect value (E) indicates the number of hits one can “expect” to see over a certain number of contiguous sequences by chance when searching a database of a certain size. The Expect value is used as a significance threshold for determining whether the hit to a database indicates true similarity. For example, an E value of 0.1 assigned to a polynucleotide hit is interpreted as meaning that in a database of the size of the EMBL database, one might expect to see 0.1 matches over the aligned portion of the sequence with a similar score simply by chance. By this criterion, the aligned and matched portions of the sequences then have a probability of 90% of being related. For sequences having an E value of 0.01 or less over aligned and matched portions, the probability of finding a match by chance in the EMBL database is 1% or less using the BLASTN algorithm. E values for polypeptide sequences may be determined in a similar fashion using various polypeptide databases, such as the SwissProt database.

According to one embodiment, “variant” polynucleotides and polypeptides, with reference to each of the polynucleotides and polypeptides of the present invention, preferably comprise sequences having the same number or fewer nucleotides or amino acids than each of the polynucleotides or polypeptides of the present invention and producing an E value of 0.01 or less when compared to the polynucleotide or polypeptide of the present invention. That is, a variant polynucleotide or polypeptide is any sequence that has at least a 99% probability of being related to the polynucleotide or polypeptide of the present invention, measured as having an E value of 0.01 or less using the BLASTN or BLASTX algorithms set at the default parameters. According to a preferred embodiment, a variant polynucleotide is a sequence having the same number or fewer nucleic acids than a polynucleotide of the present invention that has at least a 99% probability of being related to the polynucleotide of the present invention, measured as having an E value of 0.01 or less using the BLASTN algorithm set at the default parameters. Similarly, according to a preferred embodiment, a variant polypeptide is a sequence having the same number or fewer amino acids than a polypeptide of the present invention that has at least a 99% probability of being related as the polypeptide of the present invention, measured as having an E value of 0.01 or less using the BLASTP algorithm set at the default parameters.

In an alternative embodiment, variant polynucleotides are sequences that hybridize to a polynucleotide of the present invention under stringent conditions. Stringent hybridization conditions for determining complementarity include salt conditions of less than about 1 M, more usually less than about 500 mM, and preferably less than about 200 mM. Hybridization temperatures can be as low as 5° C., but are generally greater than about 22° C., more preferably greater than about 30° C., and most preferably greater than about 37° C. Longer DNA fragments may require higher hybridization temperatures for specific hybridization. Since the stringency of hybridization may be affected by other factors such as probe composition, presence of organic solvents, and extent of base mismatching, the combination of parameters is more important than the absolute measure of any one alone. An example of “stringent conditions” is prewashing in a solution of 6×SSC, 0.2% SDS; hybridizing at 65° C., 6×SSC, 0.2% SDS overnight; followed by two washes of 30 minutes each in 1×SSC, 0.1% SDS at 65° C. and two washes of 30 minutes each in 0.2×SSC, 0.1% SDS at 65° C.

The present invention also encompasses polynucleotides that differ from the disclosed sequences but that, as a consequence of the discrepancy of the genetic code, encode a polypeptide having similar enzymatic activity to a polypeptide encoded by a polynucleotide of the present invention. Thus, polynucleotides comprising sequences that differ from the polynucleotide sequences recited in SEQ ID NO: 1-4, or complements, reverse sequences, or reverse complements of those sequences, as a result of conservative substitutions are contemplated by and encompassed within the present invention. Additionally, polynucleotides comprising sequences that differ from the polynucleotide sequences recited in SEQ ID NO: 1-4, or complements, reverse complements or reverse sequences thereof, as a result of deletions and/or insertions totaling less than 10% of the total sequence length are also contemplated by and encompassed within the present invention. Similarly, polypeptides comprising sequences that differ from the polypeptide sequences recited in SEQ ID NO: 5-8 and 13-15 as a result of amino acid substitutions, insertions, and/or deletions totaling less than 10% of the total sequence length are contemplated by and encompassed within the present invention, provided the variant polypeptide has functional properties which are substantially the same as, or substantially similar to those of a polypeptide comprising a sequence of SEQ ID NO: 5-8 and 13-15.

Polynucleotides of the present invention also comprehend polynucleotides comprising at least a specified number of contiguous residues (x-mers) of any of the polynucleotides identified as SEQ ID NO: 1-4 and 9, complements, reverse sequences, and reverse complements of such sequences, and their variants. Similarly, polypeptides of the present invention comprehend polypeptides comprising at least a specified number of contiguous residues (x-mers) of any of the polypeptides identified as SEQ ID NO: 5-8, 13-15, and their variants. As used herein, the term “x-mer,” with reference to a specific value of “x,” refers to a sequence comprising at least a specified number (“x”) of contiguous residues of any of the polynucleotides identified as SEQ ID NO: 1-4 and 9, or the polypeptides identified as SEQ ID NO: 5-8 and 13-15. According to preferred embodiments, the value of x is preferably at least 20, more preferably at least 40, more preferably yet at least 60, and most preferably at least 80. Thus, polynucleotides and polypeptides of the present invention comprise a 20-mer, a 40-mer, a 60-mer, an 80-mer, a 100-mer, a 120-mer, a 150-mer, a 180-mer, a 220-mer, a 250-mer, a 300-mer, 400-mer, 500-mer or 600-mer of a polynucleotide or polypeptide identified as SEQ ID NO: 1-9, 13-15, and variants thereof.

The inventive polynucleotides may be isolated by high throughput sequencing of cDNA libraries prepared from lymph node stromal cells of fsn −/− mice as described below in Example 1. Alternatively, oligonucleotide probes based on the sequences provided in SEQ ID NO: 1-4 and 9 can be synthesized and used to identify positive clones in either cDNA or genomic DNA libraries from lymph node stromal cells of fsn −/− mice by means of hybridization or polymerase chain reaction (PCR) techniques. Probes can be shorter than the sequences provided herein but should be at least about 10, preferably at least about 15 and most preferably at least about 20 nucleotides in length. Hybridization and PCR techniques suitable for use with such oligonucleotide probes are well known in the art (see, for example, Mullis et al., Cold Spring Harbor Symp. Quant. Biol., 51:263, 1987; Erlich ed., PCR Technology, Stockton Press, NY, 1989; Sambrook et al., Molecular cloning—a laboratory manual, Cold Spring Harbor Laboratory Press, Cold Spring Harbor, N.Y., 1989). Positive clones may be analyzed by restriction enzyme digestion, DNA sequencing or the like.

The polynucleotides of the present invention may alternatively be synthesized using techniques that are well known in the art. The polynucleotides may be synthesized, for example, using automated oligonucleotide synthesizers (e.g., Beckman Oligo 1000M DNA Synthesizer) to obtain polynucleotide segments of up to 50 or more nucleic acids. A plurality of such polynucleotide segments may then be ligated using standard DNA manipulation techniques that are well known in the art of molecular biology. One conventional and exemplary polynucleotide synthesis technique involves synthesis of a single stranded polynucleotide segment having, for example, 80 nucleic acids, and hybridizing that segment to a synthesized complementary 85 nucleic acid segment to produce a 5 nucleotide overhang. The next segment may then be synthesized in a similar fashion, with a 5 nucleotide overhang on the opposite strand. The “sticky” ends ensure proper ligation when the two portions are hybridized. In this way, a complete polynucleotide of the present invention may be synthesized entirely in vitro.

Polypeptides of the present invention may be produced recombinantly by inserting a DNA sequence that encodes the polypeptide into an expression vector and expressing the polypeptide in an appropriate host. Any of a variety of expression vectors known to those of ordinary skill in the art may be employed. Expression may be achieved in any appropriate host cell that has been transformed or transfected with an expression vector containing a DNA molecule that encodes a recombinant polypeptide. Suitable host cells include prokaryotes, yeast and higher eukaryotic cells. Preferably, the host cells employed are E. coli, insect, yeast or a mammalian cell line such as COS or CHO. The DNA sequences expressed in this manner may encode naturally occurring polypeptides, portions of naturally occurring polypeptides, or other variants thereof.

In a related aspect, polypeptides are provided that comprise at least a functional portion of a polypeptide having an amino acid sequence selected from the group consisting of sequences provided in SEQ ID NO: 5-8, 13-15, and variants thereof. As used herein, the “functional portion” of a polypeptide is that portion which contains the active site essential for affecting the function of the polypeptide, for example, the portion of the molecule that is capable of binding one or more reactants. The active site may be made up of separate portions present on one or more polypeptide chains and will generally exhibit high binding affinity. Such functional portions generally comprise at least about 5 amino acid residues, more preferably at least about 10, and most preferably at least about 20 amino acid residues. Functional portions of the inventive polypeptides may be identified by first preparing fragments of the polypeptide, by either chemical or enzymatic digestion of the polypeptide or mutation analysis of the polynucleotide that encodes for the polypeptide, and subsequently expressing the resultant mutant polypeptides. The polypeptide fragments or mutant polypeptides are then tested to determine which portions retain the biological activity of the full-length polypeptide.

Portions and other variants of the inventive polypeptides may be generated by synthetic or recombinant means. Synthetic polypeptides having fewer than about 100 amino acids, and generally fewer than about 50 amino acids, may be generated using techniques well known to those of ordinary skill in the art. For example, such polypeptides may be synthesized using any of the commercially available solid-phase techniques, such as the Merrifield solid-phase synthesis method, where amino acids are sequentially added to a growing amino acid chain (Merrifield, J. Am. Chem. Soc. 85:2149-2154, 1963). Equipment for automated synthesis of polypeptides is available from suppliers such as Perkin Elmer/Applied BioSystems, Inc. (Foster City, Calif.), and may be operated according to the manufacturer's instructions. Variants of a native polypeptide may be prepared using standard mutagenesis techniques, such as oligonucleotide-directed site-specific mutagenesis (see, for example, Kunkel, Proc. Natl. Acad. Sci. USA 82:488-492, 1985). Sections of DNA sequence may also be removed using standard techniques to permit preparation of truncated polypeptides.

The present invention also provides fusion proteins comprising a first and a second inventive polypeptide or, alternatively, a polypeptide of the present invention and a known polypeptide, together with variants of such fusion proteins. The fusion proteins of the present invention may include a linker peptide between the first and second polypeptides.

A polynucleotide encoding a fusion protein of the present invention is constructed using known recombinant DNA techniques to assemble separate polynucleotides encoding the first and second polypeptides into an appropriate expression vector. The 3′ end of a polynucleotide encoding the first polypeptide is ligated, with or without a peptide linker, to the 5′ end of a DNA sequence polynucleotide encoding the second polypeptide so that the reading frames of the sequences are in phase to permit mRNA translation of the two polynucleotides into a single fusion protein that retains the biological activity of both the first and the second polypeptides.

A peptide linker sequence may be employed to separate the first and the second polypeptides by a distance sufficient to ensure that each polypeptide folds into its secondary and tertiary structures. Such a peptide linker sequence is incorporated into the fusion protein using standard techniques well known in the art. Suitable peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Preferred peptide linker sequences contain Gly, Asn and Ser residues. Other near neutral amino acids, such as Thr and Ala may also be used in the linker sequence. Amino acid sequences which may be usefully employed as linkers include those disclosed in Maratea et al., Gene 40:39-46, 1985; Murphy et al., Proc. Natl. Acad. Sci. USA 83:8258-8262, 1986; U.S. Pat. Nos. 4,935,233 and 4,751,180. The linker sequence may be from 1 to about 50 amino acids in length. Peptide linker sequences are not required when the first and second polypeptides have non-essential N-terminal amino acid regions that can be used to separate the functional domains and prevent steric interference.

The ligated polynucleotides encoding the fusion proteins are cloned into suitable expression systems using techniques known to those of ordinary skill in the art.

The polynucleotide sequences of the present invention encode polypeptides that have important role(s) in growth and development of the immune system, and in responses of the immune system to tissue injury and inflammation as well as other disease states. Some of the polynucleotides contain sequences that code for signal sequences, or transmembrane domains, which identify the protein products as secreted molecules or receptors. The polypeptides of SEQ ID NO: 5-8 have more than 25% identity to members of the fibroblast growth factor (FGF) receptor family of proteins. The inventive polypeptides have important roles in processes such as: modulation of immune responses; differentiation of precursor immune cells into specialized cell types; cell migration; cell proliferation and cell-cell interaction. The polypeptides are important in the defence of the body against infectious agents, and thus important in maintaining a disease-free environment. These polypeptides act as modulators of skin cells, especially since immune cells infiltrate skin during tissue insult, causing growth and differentiation of skin cells. In addition, these polypeptides are immunologically active, making them important therapeutic targets in a large range of disease states.

In one aspect, the present invention provides methods for using one or more of the inventive polypeptides or polynucleotides to treat a disorder in a patient. As used herein, a “patient” refers to any warm-blooded animal, preferably a human.

In this aspect, the polypeptide or polynucleotide is generally present within a composition, such as a pharmaceutical or immunogenic composition. Pharmaceutical compositions may comprise one or more polypeptides, each of which may contain one or more of the above sequences (or variants thereof), and a physiologically acceptable carrier. Immunogenic compositions may comprise one or more of the above polypeptides and an immunostimulant, such as an adjuvant or a liposome, into which the polypeptide is incorporated.

Alternatively, a composition of the present invention may contain DNA encoding one or more polypeptides described above, such that the polypeptide is generated in situ. In such compositions, the DNA may be present within any of a variety of delivery systems known to those of ordinary skill in the art, including nucleic acid expression systems, and bacterial and viral expression systems. Appropriate nucleic acid expression systems contain the necessary DNA sequences for expression in the patient (such as a suitable promoter and terminator signal). Bacterial delivery systems involve the administration of a bacterium (such as Bacillus Calnette-Guerin) that expresses an immunogenic portion of the polypeptide on its cell surface. In a preferred embodiment, the DNA may be introduced using a viral expression system (e.g., vaccinia or other poxvirus, retrovirus, or adenovirus), which may involve the use of a non-pathogenic, or defective, replication competent virus. Techniques for incorporating DNA into such expression systems are well known in the art. The DNA may also be “naked,” as described, for example, in Ulmer et al., Science 259:1745-1749, 1993 and reviewed by Cohen, Science 259:1691-1692, 1993. The uptake of naked DNA may be increased by coating the DNA onto biodegradable beads, which are efficiently transported into the cells.

Routes and frequency of administration, as well as dosage, vary from individual to individual. In general, the inventive compositions may be administered by injection (e.g., intradermal, intramuscular, intravenous or subcutaneous), intranasally (e.g., by aspiration) or orally. In general, the amount of polypeptide present in a dose (or produced in situ by the DNA in a dose) ranges from about 1 μg to about 100 mg per kg of host, typically from about 10 μg to about 1 mg per kg of host, and preferably from about 100 μg to about 1 μg per kg of host. Suitable dose sizes will vary with the size of the patient, but will typically range from about 0.1 ml to about 2 ml.

While any suitable carrier known to those of ordinary skill in the art may be employed in the pharmaceutical compositions of this invention, the type of carrier will vary depending on the mode of administration. For parenteral administration, such as subcutaneous injection, the carrier preferably comprises water, saline, alcohol, a lipid, a wax or a buffer. For oral administration, any of the above carriers or a solid carrier, such as mannitol, lactose, starch, magnesium stearate, sodium saccharine, talcum, cellulose, glucose, sucrose, and magnesium carbonate, may be employed. Biodegradable microspheres (e.g., polylactic galactide) may also be employed as carriers for the pharmaceutical compositions of this invention. Suitable biodegradable microspheres are disclosed, for example, in U.S. Pat. Nos. 4,897,268 and 5,075,109.

Any of a variety of adjuvants may be employed in the immunogenic compositions of the present invention to non-specifically enhance the immune response. Most adjuvants contain a substance designed to protect the antigen from rapid catabolism, such as aluminum hydroxide or mineral oil, and a non-specific stimulator of immune responses, such as lipid A, Bordetella pertussis or M. tuberculosis. Suitable adjuvants are commercially available as, for example, Freund's Incomplete Adjuvant and Freund's Complete Adjuvant (Difco Laboratories, Detroit, Mich.), and Merck Adjuvant 65 (Merck and Company, Inc., Rahway, N.J.). Other suitable adjuvants include alum, biodegradable microspheres, monophosphoryl lipid A and Quil A.

The polynucleotides of the present invention may also be used as markers for tissue, as chromosome markers or tags, in the identification of genetic disorders, and for the design of oligonucleotides for examination of expression patterns using techniques well known in the art, such as the microarray technology available from Affymetrix (Santa Clara, Calif.). Partial polynucleotide sequences disclosed herein may be employed to obtain full length genes by, for example, screening of DNA expression libraries, and to isolate homologous DNA sequences from other species using hybridization probes or PCR primers based on the inventive sequences.

The isolated polynucleotides of the present invention also have utility in genome mapping, in physical mapping, and in positional cloning of genes. As detailed below, the polynucleotide sequences identified as SEQ ID NO: 1-4, and their variants, may be used to design oligonucleotide probes and primers. Oligonucleotide probes designed using the polynucleotides of the present invention may be used to detect the presence and examine the expression patterns of genes in any organism having sufficiently similar DNA and RNA sequences in their cells using techniques that are well known in the art, such as slot blot DNA hybridization techniques. Oligonucleotide primers designed using the polynucleotides of the present invention may be used for PCR amplifications. Oligonucleotide probes and primers designed using the polynucleotides of the present invention may also be used in connection with various microarray technologies, including the microarray technology of Affymetrix (Santa Clara, Calif.).

As used herein, the term “oligonucleotide” refers to a relatively short segment of a polynucleotide sequence, generally comprising between 6 and 60 nucleotides, and comprehends both probes for use in hybridization assays and primers for use in the amplification of DNA by polymerase chain reaction. An oligonucleotide probe or primer is described as “corresponding to” a polynucleotide of the present invention, including one of the sequences set out as SEQ ID NO: 1-4 and 9, or a variant thereof, if the oligonucleotide probe or primer, or its complement, is contained within one of the sequences set out as SEQ ID NO: 1-4 and 9, or a variant of one of the specified sequences. Oligonucleotide probes and primers of the present invention are substantially complementary to a polynucleotide disclosed herein.

Two single stranded sequences are said to be substantially complementary when the nucleotides of one strand, optimally aligned and compared, with the appropriate nucleotide insertions and/or deletions, pair with at least 80%, preferably at least 90% to 95% and more preferably at least 98% to 100% of the nucleotides of the other strand. Alternatively, substantial complementarity exists when a first DNA strand will selectively hybridize to a second DNA strand under stringent hybridization conditions. Stringent hybridization conditions for determining complementarity include salt conditions of less than about 1 M, more usually less than about 500 mM, and preferably less than about 200 mM. Hybridization temperatures can be as low as 5° C., but are generally greater than about 22° C., more preferably greater than about 30° C., and most preferably greater than about 37° C. Longer DNA fragments may require higher hybridization temperatures for specific hybridization. Since the stringency of hybridization may be affected by other factors such as probe composition, presence of organic solvents and extent of base mismatching, the combination of parameters is more important than the absolute measure of any one alone.

In specific embodiments, the oligonucleotide probes and/or primers comprise at least about 6 contiguous residues, more preferably at least about 10 contiguous residues, and most preferably at least about 20 contiguous residues complementary to a polynucleotide sequence of the present invention. Probes and primers of the present invention may be from about 8 to 100 base pairs in length or, preferably from about 10 to 50 base pairs in length or, more preferably from about 15 to 40 base pairs in length. The probes can be easily selected using procedures well known in the art, taking into account DNA-DNA hybridization stringencies, annealing and melting temperatures, and potential for formation of loops and other factors, which are well known in the art. Tools and software suitable for designing probes and PCR primers are well known in the art and include the software program available from Premier Biosoft International, 3786 Corina Way, Palo Alto, Calif. 94303-4504. Preferred techniques for designing PCR primers are also disclosed in Dieffenbach, C W and Dyksler, G S. PCR Primer: a laboratory manual, CSHL Press: Cold Spring Harbor, N.Y., 1995.

A plurality of oligonucleotide probes or primers corresponding to a polynucleotide of the present invention may be provided in a kit form. Such kits generally comprise multiple DNA or oligonucleotide probes or primers, each probe or primer being specific for a polynucleotide sequence. Kits of the present invention may comprise one or more probes or primers corresponding to a polynucleotide of the present invention, including a polynucleotide sequence identified in SEQ ID NO: 1-4 and 9.

In one embodiment useful for high-throughput assays, the oligonucleotide probe kits of the present invention comprise multiple probes in an array format, wherein each probe is immobilized at a predefined, spatially addressable, location on the surface of a solid substrate. Array formats which may be usefully employed in the present invention are disclosed, for example, in U.S. Pat. Nos. 5,412,087 and 5,545,451, and PCT Publication No. WO 95/00450, the disclosures of which are hereby incorporated by reference.

The polynucleotides of the present invention may also be used to tag or identify an organism or reproductive material therefrom. Such tagging may be accomplished, for example, by stably introducing a non-disruptive non-functional heterologous polynucleotide identifier into an organism, the polynucleotide comprising one of the polynucleotides of the present invention.

The polypeptides provided by the present invention may additionally be used in assays to determine biological activity, to raise antibodies, to isolate corresponding ligands or receptors, in assays to quantify levels of protein or cognate corresponding ligand or receptor, as anti-inflammatory agents, and in compositions for the treatment of diseases of the immune system.

The present invention further provides methods and compositions for reducing the levels and/or inhibiting the activity of an inventive polypeptide or polynucleotide. Such methods include administering a component selected from the group consisting of: antibodies, or antigen-binding fragments thereof, that specifically bind to a polypeptide of the present invention; soluble ligands that bind to an inventive polypeptide; small molecule inhibitors of the inventive polypeptides and/or polynucleotides; anti-sense oligonucleotides to the inventive polynucleotides; small interfering RNA molecules (siRNA or RNAi) that are specific for a polynucleotide or polypeptide of the present invention; and engineered soluble polypeptide molecules that bind a ligand of an inventive polypeptide but do not stimulate signaling.

Modulating the activity of a polypeptide described herein may be accomplished by reducing or inhibiting expression of the polypeptides, which can be achieved by interfering with transcription and/or translation of the corresponding polynucleotide. Polypeptide expression may be inhibited, for example, by introducing anti-sense expression vectors; by introducing anti-sense oligodeoxyribonucleotides, anti-sense phosphorothioate oligodeoxyribonucleotides, anti-sense oligoribonucleotides or anti-sense phosphorothioate oligoribonucleotides; or by other means well known in the art. All such anti-sense polynucleotides are referred to collectively herein as “anti-sense oligonucleotides”.

The anti-sense oligonucleotides disclosed herein are sufficiently complementary to the polynucleotide encoding the inventive polypeptide to bind specifically to the polynucleotide. The sequence of an anti-sense oligonucleotide need not be 100% complementary to that of the polynucleotide in order for the anti-sense oligonucleotide to be effective in the inventive methods. Rather an anti-sense oligonucleotide is sufficiently complementary when binding of the anti-sense oligonucleotide to the polynucleotide interferes with the normal function of the polynucleotide to cause a loss of utility, and when non-specific binding of the oligonucleotide to other, non-target, sequences is avoided. The present invention thus encompasses polynucleotides in an anti-sense orientation that inhibit translation of the inventive polypeptides. The design of appropriate anti-sense oligonucleotides is well known in the art. Oligonucleotides that are complementary to the 5′ end of the message, for example the 5′ untranslated sequence up to and including the AUG initiation codon, should work most efficiently at inhibiting translation. However, oligonucleotides complementary to either the 5′- or 3′-non-translated, non-coding, regions of the targeted polynucleotide can be used.

Cell permeation and activity of anti-sense oligonucleotides can be enhanced by appropriate chemical modifications, such as the use of phenoxazine-substituted C-5 propynyl uracil oligonucleotides (Flanagan et al., Nat. Biotechnol. 17:48-52 (1999)) or 2′-O-(2-methoxy) ethyl (2′-MOE)-oligonucleotides (Zhang et al., Nat. Biotechnol. 18:862-867 (2000)). The use of techniques involving anti-sense oligonucleotides is well known in the art and is described, for example, in Robinson-Benion et al., Methods in Enzymol. 254:363-375 (1995) and Kawasaki et al., Artific. Organs 20:836-848 (1996).

Expression of a polypeptide of the present invention may also be specifically suppressed by methods such as RNA interference (RNAi). A review of this technique is found in Science, 288:1370-1372, 2000. Briefly, traditional methods of gene suppression, employing anti-sense RNA or DNA, operate by binding to the reverse sequence of a gene of interest such that binding interferes with subsequent cellular processes and therefore blocks synthesis of the corresponding protein. RNAi also operates on a post-translational level and is sequence specific, but suppresses gene expression far more efficiently. Exemplary methods for controlling or modifying gene expression are provided in WO 99/49029, WO 99/53050 and WO0/75164, the disclosures of which are hereby incorporated by reference. In these methods, post-transcriptional gene silencing is brought about by a sequence-specific RNA degradation process which results in the rapid degradation of transcripts of sequence-related genes. Studies have shown that double-stranded RNA may act as a mediator of sequence-specific gene silencing (see, for example, Montgomery and Fire, Trends in Genetics, 14:255-258, 1998). Gene constructs that produce transcripts with self-complementary regions are particularly efficient at gene silencing.

It has been demonstrated that one or more ribonucleases specifically bind to and cleave double-stranded RNA into short fragments. The ribonuclease(s) remains associated with these fragments, which in turn specifically bind to complementary mRNA, i.e. specifically bind to the transcribed mRNA strand for the gene of interest. The mRNA for the gene is also degraded by the ribonuclease(s) into short fragments, thereby obviating translation and expression of the gene. Additionally, an RNA-polymerase may act to facilitate the synthesis of numerous copies of the short fragments, which exponentially increases the efficiency of the system. A unique feature of RNAi is that silencing is not limited to the cells where it is initiated. The gene-silencing effects may be disseminated to other parts of an organism.

The polynucleotides of the present invention may thus be employed to generate gene silencing constructs and/or gene-specific self-complementary, double-stranded RNA sequences that can be delivered by conventional art-known methods. A gene construct may be employed to express the self-complementary RNA sequences. Alternatively, cells are contacted with gene-specific double-stranded RNA molecules, such that the RNA molecules are internalized into the cell cytoplasm to exert a gene silencing effect. The double-stranded RNA must have sufficient homology to the targeted gene to mediate RNAi without affecting expression of non-target genes. The double-stranded DNA is at least 20 nucleotides in length, and is preferably 21-23 nucleotides in length. Preferably, the double-stranded RNA corresponds specifically to a polynucleotide of the present invention. The use of small interfering RNA (siRNA) molecules of 21-23 nucleotides in length to suppress gene expression in mammalian cells is described in WO 01/75164. Tools for designing optimal inhibitory siRNAs include that available from DNAengine Inc. (Seattle, Wash.).

One RNAi technique employs genetic constructs within which sense and anti-sense sequences are placed in regions flanking an intron sequence in proper splicing orientation with donor and acceptor splicing sites. Alternatively, spacer sequences of various lengths may be employed to separate self-complementary regions of sequence in the construct. During processing of the gene construct transcript, intron sequences are spliced-out, allowing sense and anti-sense sequences, as well as splice junction sequences, to bind forming double-stranded RNA. Select ribonucleases then bind to and cleave the double-stranded RNA, thereby initiating the cascade of events leading to degradation of specific mRNA gene sequences, and silencing specific genes.

As used herein, the phrase “contacting a population of cells with a genetic construct, anti-sense oligonucleotide or RNA molecule” includes any means of introducing a nucleic acid molecule into any portion of one or more cells by any method compatible with cell viability and known to those of ordinary skill in the art. The cell or cells may be contacted in vivo, ex vivo, in vitro, or any combination thereof.

For in vivo uses, a genetic construct, anti-sense oligonucleotide or RNA molecule may be administered by various art-recognized procedures. See, e.g., Rolland, Crit. Rev. Therap. Drug Carrier Systems 15:143-198 (1998), and cited references. Both viral and non-viral delivery methods have been used for gene therapy. Useful viral vectors include, for example, adenovirus, adeno-associated virus (AAV), retrovirus, vaccinia virus and avian poxvirus. Improvements have been made in the efficiency of targeting genes to tumor cells with adenoviral vectors, for example, by coupling adenovirus to DNA-polylysine complexes and by strategies that exploit receptor-mediated endocytosis for selective targeting. See, e.g., Curiel et al., Hum. Gene Ther., 3:147-154 (1992); and Cristiano and Curiel, Cancer Gene Ther. 3:49-57 (1996). Non-viral methods for delivering polynucleotides are reviewed in Chang & Seymour, (Eds) Curr. Opin. Mol. Ther., vol. 2 (2000). These methods include contacting cells with naked DNA, cationic liposomes, or polyplexes of polynucleotides with cationic polymers and dendrimers for systemic administration (Chang & Seymour, Ibid.). Liposomes can be modified by incorporation of ligands that recognize cell-surface receptors and allow targeting to specific receptors for uptake by receptor-mediated endocytosis. See, for example, Xu et al., Mol. Genet. Metab., 64:193-197 (1998); and Xu et al., Hum. Gene Ther., 10:2941-2952 (1999).

Tumor-targeting bacteria, such as Salmonella, are potentially useful for delivering genes to tumors following systemic administration (Low et al., Nat. Biotechnol. 17:37-41 (1999)). Bacteria can be engineered ex vivo to penetrate and to deliver DNA with high efficiency into mammalian epithelial cells in vivo and in vitro. See, e.g., Grillot-Courvalin et al., Nat. Biotechnol. 16:862-866 (1998). Degradation-stabilized oligonucleotides may be encapsulated into liposomes and delivered to patients by injection either intravenously or directly into a target site. Alternatively, retroviral or adenoviral vectors, or naked DNA expressing anti-sense RNA for the inventive polypeptides, may be delivered into patient's cells in vitro or directly into patients in vivo by appropriate routes. Suitable techniques for use in such methods are well known in the art.

The present invention further provides binding agents, such as antibodies and antigen-binding fragments thereof, that specifically bind to a polypeptide disclosed herein, or to a portion or variant thereof. A binding agent is said to “specifically bind” to an inventive polypeptide if it reacts at a detectable level with the polypeptide, and does not react detectably with unrelated polypeptides under similar conditions Any agent that satisfies this requirement may be a binding agent. For example, a binding agent may be a ribosome, with or without a peptide component, an RNA molecule, or a polypeptide. In a preferred embodiment, a binding agent is an antibody or an antigen-binding fragment thereof. The ability of an antibody, or antigen-binding fragment thereof, to specifically bind to a polypeptide can be determined, for example, in an ELISA assay using techniques well known in the art.

An “antigen-binding site,” or “antigen-binding fragment” of an antibody refers to the part of the antibody that participates in antigen binding. The antigen binding site is formed by amino acid residues of the N-terminal variable (“V”) regions of the heavy (“H”) and light (“L”) chains. Three highly divergent stretches within the V regions of the heavy and light chains are referred to as “hypervariable regions” which are interposed between more conserved flanking stretches known as “framework regions,” or “FRs”. Thus the term “FR” refers to amino acid sequences which are naturally found between and adjacent to hypervariable regions in immunoglobulins. In an antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.”

Antibodies may be prepared by any of a variety of techniques known to those of ordinary skill in the art. See, e.g., Harlow and Lane, Antibodies: A Laboratory Manual, Cold Spring Harbor Laboratory, 1988. In general, antibodies can be produced by cell culture techniques, including the generation of monoclonal antibodies as described herein, or via transfection of antibody genes into suitable bacterial or mammalian cell hosts, in order to allow for the production of recombinant antibodies. In one technique, an immunogen comprising the inventive polypeptide is initially injected into any of a wide variety of mammals (e.g., mice, rats, rabbits, sheep or goats). The polypeptides of this invention may serve as the immunogen without modification. Alternatively, particularly for relatively short polypeptides, a superior immune response may be elicited if the polypeptide is joined to a carrier protein, such as bovine serum albumin or keyhole limpet hemocyanin. The immunogen is injected into the animal host, preferably according to a predetermined schedule incorporating one or more booster immunizations, and the animals are bled periodically. Polyclonal antibodies specific for the inventive polypeptide may then be purified from such antisera by, for example, affinity chromatography using the polypeptide coupled to a suitable solid support.

Monoclonal antibodies specific for an inventive polypeptide may be prepared using the technique of Kohler and Milstein, Eur. J. Immunol. 6:511-519, 1976, and improvements thereto. These methods involve the preparation of immortal cell lines capable of producing antibodies having the desired specificity. Such cell lines may be produced from spleen cells obtained from an animal immunized as described above. The spleen cells are then immortalized by, for example, fusion with a myeloma cell fusion partner, preferably one that is syngeneic with the immunized animal. A variety of fusion techniques well known in the art may be employed. For example, the spleen cells and myeloma cells may be combined with a nonionic detergent for a few minutes and then plated at low density on a selective medium that supports the growth of hybrid cells, but not myeloma cells. A preferred selection technique uses HAT (hypoxanthine, aminopterin, thymidine) selection. After a sufficient time, usually about 1 to 2 weeks, colonies of hybrids are observed. Single colonies are selected and their culture supernatants tested for binding activity against the polypeptide. Hybridomas having high reactivity and specificity are preferred.

Monoclonal antibodies may then be isolated from the supernatants of growing hybridoma colonies. In addition, various techniques may be employed to enhance the yield, such as injection of the hybridoma cell line into the peritoneal cavity of a suitable vertebrate host, such as a mouse. Monoclonal antibodies may then be harvested from the ascites fluid or the blood. Contaminants may be removed from the antibodies by conventional techniques, such as chromatography, gel filtration, precipitation, and extraction. The polypeptides of this invention may be used in the purification process in, for example, an affinity chromatography step.

A number of molecules are known in the art that comprise antigen-binding sites capable of exhibiting the binding properties of an antibody molecule. For example, the proteolytic enzyme papain preferentially cleaves IgG molecules to yield several fragments, two of which (the “F(ab)” fragments) each comprise a covalent heterodimer that includes an intact antigen-binding site. The enzyme pepsin is able to cleave IgG molecules to provide several fragments, including the “F(ab′)₂” fragment, which comprises both antigen-binding sites. An “Fv” fragment can be produced by preferential proteolytic cleavage of an IgM, IgG or IgA immunoglobulin molecule, but are more commonly derived using recombinant techniques known in the art. The Fv fragment includes a non-covalent V_{H}::V_{L} heterodimer including an antigen-binding site which retains much of the antigen recognition and binding capabilities of the native antibody molecule (Inbar et al. Proc. Nat. Acad. Sci. USA 69:2659-2662 (1972); Hochman et al. Biochem 15:2706-2710 (1976); and Ehrlich et al. Biochem 19:4091-4096 (1980)).

The present invention further encompasses humanized antibodies that specifically bind to an inventive polypeptide. A number of humanized antibody molecules comprising an antigen-binding site derived from a non-human immunoglobulin have been described, including chimeric antibodies having rodent V regions and their associated CDRs fused to human constant domains (Winter et al. Nature 349:293-299 (1991); Lobuglio et al. Proc. Nat. Acad. Sci. USA 86:4220-4224 (1989); Shaw et al. J Immunol. 138:4534-4538 (1987); and Brown et al. Cancer Res. 47:3577-3583 (1987)); rodent CDRs grafted into a human supporting FR prior to fusion with an appropriate human antibody constant domain (Riechmann et al. Nature 332:323-327 (1988); Verhoeyen et al. Science 239:1534-1536 (1988); and Jones et al. Nature 321:522-525 (1986)); and rodent CDRs supported by recombinantly veneered rodent FRs (European Patent Publication No. 519,596, published Dec. 23, 1992). These “humanized” molecules are designed to minimize unwanted immunological responses towards rodent antihuman antibody molecules which limit the duration and effectiveness of therapeutic applications of those moieties in human recipients.

### EXAMPLE 1

### Isolation of cDNA Sequences from Lymph Node Stromal Cell Expression Libraries

The cDNA sequences of the present invention were obtained by high-throughput sequencing of cDNA expression libraries constructed from murine fsn −/− lymph node stromal cells as described below.

cDNA Libraries from Lymph Node Stromal Cells (MLSA and MLSE)

Lymph nodes were removed from flaky skin fsn −/− mice, the cells dissociated and the resulting single cell suspension placed in culture. After four passages, the cells were harvested. Total RNA, isolated using TRIzol Reagent (BRL Life Technologies, Gaithersburg, Md.), was used to obtain mRNA using a Poly(A) Quik mRNA isolation kit (Stratagene, La Jolla, Calif.), according to the manufacturer's specifications. A cDNA expression library (referred to as the MLSA library) was then prepared from the mRNA by Reverse Transcriptase synthesis using a Lambda ZAP Express cDNA library synthesis kit (Stratagene, La Jolla, Calif.). A second cDNA expression library, referred to as the MLSE library, was prepared exactly as above except that the cDNA was inserted into the mammalian expression vector pcDNA3 (Invitrogen, Carlsbad Calif.).

The nucleotide sequence of a cDNA clone isolated from the MLSA library is given in SEQ ID NO: 1, with the corresponding amino acid sequence being provided in SEQ ID NO: 5.

### EXAMPLE 2

### Characterization of Isolated cDNA Sequences

The isolated cDNA sequences were compared to sequences in the EMBL DNA database using the computer algorithm BLASTN, and the corresponding polypeptide sequences (DNA translated to protein in each of 6 reading frames) were compared to sequences in the SwissProt database using the computer algorithm BLASTP. Specifically, comparisons of DNA sequences provided in SEQ ID NO: 1-4 to sequences in the EMBL (Release 60, September 1999) DNA database, and amino acid sequences provided in SEQ ID NO: 5-8 to sequences in the SwissProt and TrEMBL (up to Oct. 20, 1999) databases were made as of Dec. 31, 1999. The cDNA sequences of SEQ ID NO: 1-4, and their corresponding polypeptide sequences (SEQ ID NO: 5-8, respectively) were determined to have less than 75% identity (determined as described above) to sequences in the EMBL and SwissProt databases using the computer algorithms BLASTN and BLASTP, respectively.

Using automated search programs to screen against sequences coding for known molecules reported to be of therapeutic and/or diagnostic use, the isolated polynucleotides of SEQ ID NO: 1-4 were determined to encode polypeptide sequences that are members of the fibroblast growth factor (FGF) receptor family (SEQ ID NO: 5-8). A family member is herein defined to have at least 20% identical amino acid residues in the translated polypeptide to a known protein or member of a protein family.

Fibroblast growth factor receptors belong to a family of four single membrane-spanning tyrosine kinases (FGFR1 to 4). These receptors serve as high-affinity receptors for 17 growth factors (FGF1 to 17). FGF receptors have important roles in multiple biological processes, including mesoderm induction and patterning, cell growth and migration, organ formation and bone growth (Xu, Cell Tissue Res. 296:33-43, 1999). Further analysis of the sequence revealed the presence of a putative transmembrane domain and intracellular domain, similar to other FGF receptors.

### EXAMPLE 3

### Isolation of Full Length cDNA Sequence of a Murine Fibroblast Growth Factor Receptor Homolog

The full-length cDNA sequence of a murine fibroblast growth factor receptor homolog was isolated as follows.

The MLSA cell cDNA library (described in Example 1) was screened with an [α ³²P]-dCTP labeled cDNA probe corresponding to nucleotides 1 to 451 of the coding region within SEQ ID NO: 1. Plaque lifts, hybridization and screening were performed using standard molecular biology techniques. The determined polynucleotide sequence of the full-length murine FGFR gene (referred to as muFGFR-β) is provided in SEQ ID NO: 2, with the corresponding polypeptide sequence being provided in SEQ ID NO: 6.

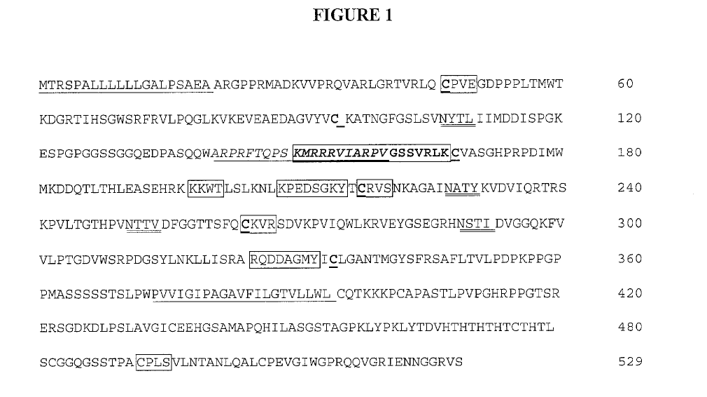

Analysis of the polynucleotide sequence of SEQ ID NO: 2 revealed the presence of a putative transmembrane domain corresponding to nucleotides 1311 to 1370. The polypeptide sequence (SEQ ID NO: 6; FIG. 1) has regions similar to the extracellular domain of the fibroblast growth factor receptor family. The amino acid sequence of the extracellular domain of muFGFR-β is provided in SEQ ID NO: 13, while the amino acid sequence of the intracellular domain is provided in SEQ ID NO: 14.

A splice variant of SEQ ID NO: 2 was also isolated from the MLSA cDNA library as described in Example 1. The determined polynucleotide sequence of the splice variant (referred to as FGFR-γ) is provided in SEQ ID NO: 3 and the corresponding polypeptide sequence is provided in SEQ ID NO: 7. The splice regions are in an equivalent position to splice sites for previously described FGF receptors (Ornitz, J. Biol. Chem. 296:15292-15297, 1996; Wilkie, Current Biology 5:500-507, 1995; Miki, Proc. Natl. Acad. Sci. USA 89:246-250, 1992), thus establishing that this molecule (referred to as FGFR5) is a FGF receptor homolog. The main difference between the two FGFR5 splice variants is that muFGFR-β contains three extracellular Ig-domains, while FGFR-γ contains only two such domains.

To examine the structural similarities between FGFR5 and the other members of the FGF receptor family, 3D Swiss modeller (Petisch, Bio/Technology 13:658-660, 1995; Peitsch, Biochem Soc Trans. 24:274-279, 1996; Guex and Peitsch, Electrophoresis 18:2714-2723, 1997) was employed to produce a predicted crystal structure of the extracellular domain of FGFR-γ. These studies showed that the crystal structure of FGFR5 deviates from that of the known FGFR1 structure between residues 188 and 219 of SEQ ID NO: 7 (SEQ ID NO: 15). These residues correlate with an area of low homology between FGFR5 and other members of the FGF receptor family that may have a critical role in defining ligand specificity.

The critical residues for ligand binding have previously been identified in co-crystallization studies of FGFR1 binding FGF-2 (Plotnikov et al., Cell 98:641-650, 1999). Alignment of FGFR-γ with FGFR1 showed that many of these residues are conserved or are a conservative substitution. Conserved ligand binding residues between the two receptors are found at residues 66, 68, 146, 178, 181, 183 and 216 of SEQ ID NO: 7, while conservative substitutions of potential ligand binding residues are found at residues 64, 180 and 226 of SEQ ID NO: 7. When visualized on the predicted crystal structure of FGFR-γ, these residues line the groove of the ligand binding domain. Thus, while the overall degree of similarity between FGFR5 and other FGF receptors is relatively low, the extracellular domains of the FGFR5 splice variants have all the conserved residues important for ligand binding.

The main difference between the FGFR5 receptor and other family members is the lack of an intracellular tyrosine kinase domain. With the four previously identified FGF receptors (FGFR1-4), signal transduction is mediated by ligand binding and receptor dimerization, resulting in autophosphorylation of the tyrosine residues within the intracellular RTK domain. This autophosphorylation then phosphorylates a number of intracellular substrates, initiating several signal transduction cascades. The FGFR5 splice variants described herein each contain tyrosine residues in the intracellular domain demonstrating similarity to a SHP binding motif (residues 458-463 of SEQ ID NO: 6 and 367-377 of SEQ ID NO: 7). SHPs are protein tyrosine phosphatases that participate in cellular signalling and that have previously been identified in the cytoplasmic domains of many receptors eliciting a broad range of activities. The presence of such motifs in the cytoplasmic domain of FGFR5 is thus indicative of signalling, and modification of these motifs may be employed to modulate signal transduction initiated by binding of a ligand to FGFR5. These motifs are conserved between the mouse transcripts of FGFR5 and the human homolog described below. Removal or modification of these signaling motifs and/or the cytoplasmic domain of FGFR5 may be employed to engineer a soluble FGFR5-like molecule that binds to the FGFR5 ligand without stimulating signaling. Such a molecule may be usefully employed to modulate the binding, and therefore activity, of FGFR5.

### EXAMPLE 4

### Isolation of a Human FGF Receptor Homolog

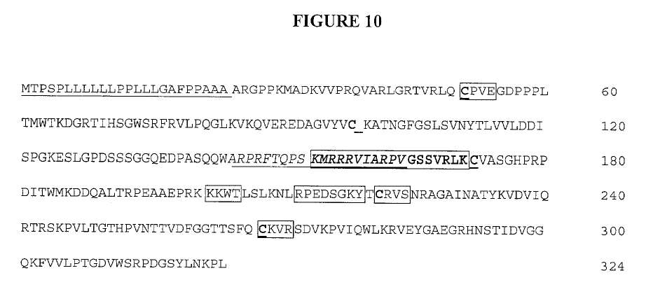

The cDNA encoding the partial murine FGF receptor (SEQ ID NO: 1) was used to search the EMBL database (Release 58, March 1999) to identify human EST homologs. The identified EST (Accession Number AI245701) was obtained from Research Genetics, Inc (Huntsville Ala.) as I.M.A.G.E. Consortium clone ID 1870593. Sequence determination of the complete insert of clone 1870593 resulted in the identification of 520 additional nucleotides. The insert of this clone did not represent the full-length gene. The determined nucleotide sequence of the complete insert of clone 1870593, which represents the extracellular domain of the human FGF receptor homolog, is given in SEQ ID NO: 4 and the polypeptide sequence of the extracellular domain in SEQ ID NO: 8. Several conserved domains were identified in SEQ ID NO: 8 that are involved in the dimerization, ligand binding and activity of the receptor. These are shown in FIG. 10.

Both murine and human FGFR5 are structurally similar to FGFR1-4, the other members of the FGFR family. In the extracellular domain, three immunoglobulin-like motifs are present that are flanked by conserved cysteine residues. The Ig-1 loop is the least conserved of the three Ig loops and is not required for ligand binding, but regulates binding affinity (Shi et al., Mol. Cell Biol. 13:3907-3918, 1993). The Ig-3 loop is involved in ligand selectivity (Omitz et al., Science 268:432-436, 1996).

An acidic box is characteristic in FGFR1-4 and is involved in binding divalent cations, including copper and calcium. Acidic boxes are important for interaction with cell adhesion molecules, extracellular matrix and heparin (Patstone and Maher, J. Biol. Chem. 271:3343-3346). The acidic box in FGFR5 is smaller than in the other four receptors or absent.

The cell adhesion molecule (CAM) homology and heparin-binding domain is also characteristic of the extracellular domain (Szebenyi and Fallon, Int. Rev. Cytol. 185:45-106). The CAM homology region is a binding site for L1, N-CAM and N-cadherin (Doherty et al., 1996).

The FGFR5 heparin-binding domain is typical of other FGFR heparin-binding domains and consists of a cluster of basic and hydrophobic residues flanked by Lys residues (Kan et al., Science 259:1918-1921, 1993). Heparin or heparan sulfate proteoglycans are essential co-factors for the interaction of FGFs with FGFRs and it has been shown that heparin is a growth-factor independent ligand for FGFR4 (Gao and Goldfarb, EMBO J. 14:2183-2190, 1995).

### EXAMPLE 5

### Characterization of the Murine FGF Receptor Homolog

Soluble forms of the murine FGF receptor homolog muFGFR-γ and splice variant FGFR-γ (SEQ ID NO: 2 and 3, respectively) were expressed in mammalian cells and the purified proteins used to determine the ligand binding specificity of the molecules as follows.

The extracellular domains of muFGFR-β and FGFR-γ were amplified by PCR using primers MS158 and MS159 (SEQ ID NO: 10 and 11, respectively) and cloned into the expression vector pcDNA3 containing the Fc fragment from human IgG1. These soluble recombinant proteins, referred to as FGFRβFc and FGFRγFc, were expressed in HEK293 cells (ATCC No. CRL-1573, American Type Culture Collection, Manassas, Va.) and purified using an Affiprep protein A column (Biorad, Hercules Calif.).

FGF-2 (basic fibroblast growth factor) has previously been demonstrated to bind all FGF receptors but with a range of different affinities. Binding of muFGFR-β to FGF-2 was demonstrated by co-incubating the purified protein and FGF-2 in the presence of protein G Sepharose (Amersham Pharmacia, Uppsala, Sweden) and resolving complexes formed on denaturing polyacrylamide gels. FGF-2 (2 μg) was incubated with 5 μg FGFRβFc, FGF Receptor 2 (FGFR2Fc) or unrelated protein (MLSA8790Fc) in 5 μl protein G fast flow beads (Pharmacia, Uppsala, Sweden), PBS and 0.1% Triton X-100 for 60 min at 4° C. The beads were washed three times in 0.1% Triton X-100/PBS and resuspended in 20 μl loading buffer (0.1 M DTT, 10% sucrose, 60 MM Tris.HCl pH 6.8, 5% SDS and 0.01% bromophenol blue). The samples were analysed on a 12% polyacrylamide gel. FGF-2, FGFR2Fc, FGFRβFc and MLSA8790Fc (1 μg of each) were loaded on the gel for comparison. After staining of the gel with Coomassie blue, a doublet of bands were visible in the lane containing FGFRβFc, indicating that a complex formed between the FGF-2 and the murine FGF receptor homolog FGFRβFc, and that FGF-2 is a ligand for the novel FGF receptor homolog. A doublet was also observed in the lane containing the FGFR2Fc, which was the positive control. No doublet was observed in the negative control lane containing the MLSA8790Fc protein.

The binding specificity of the murine FGF receptor homolog FGFRβFc was further examined by repeating the experiment described above, replacing the FGF-2 with another known growth factor, epidermal growth factor (EGF). In this experiment, EGF did not bind to FGFR2Fc, FGFRβFc or MLSAS790Fc, indicating that binding of FGF-2 to the murine FGF receptor homolog FGFRβFc was specific. Similarly, in subsequent experiments employing FGF-7, no binding of FGFR2Fc, FGFRβFc or MLSA8790Fc was observed.

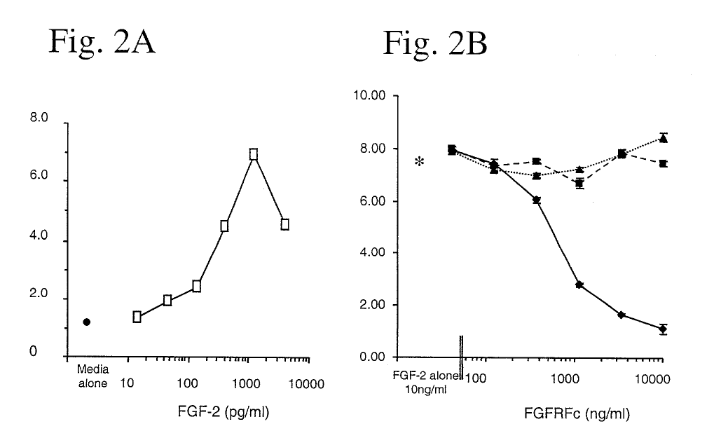

To determine the difference in binding affinity between FGFR5 and FGFR2, the ability of FGFRβFc and FGFRγFc to inhibit FGF signalling in FGF-responsive NIH-3T3 SRE reporter cells was examined. Fibroblast growth factors typically signal via phosphorylation of the receptor tyrosine kinase domain stimulating the MAP kinase pathway. This eventually leads to activation of genes under the control of the serum response element (SRE). Reporter constructs containing concatamerised SRE sequences upstream of a luciferase reporter gene were stably transfected into NIH-3T3 cells. Reporter activity was measured by measuring luciferase levels. As shown in FIG. 2A, a dose dependent response of NIH-3T3 SRE cells to FGF-2 was seen in the presence of heparin. Using a standard dose of FGF-2 in the presence of heparin, an increasing concentration of FGFR2Fc, FGFRβFc or FGFRγFc was titrated onto the NIH-3T3 SRE cells and luciferase activity was measured. Increasing concentrations of FGFR2Fc, the positive control, reduced the luciferase signal in FGF-2 stimulated cells (FIG. 2B). However, titrating FGFRβFc and FGFRγFc did not inhibit FGF-mediated luciferase signal from the NIH-3T3 SRE cells. These results show that FGF-2 has lower affinity for either FGFRβ or FGFRγ than for FGFR2, and indicate that the ligand specificity of FGFR5 is different to those of the other members of the FGF receptor family.

### EXAMPLE 6

### Sequence Determination of a Polynucleotide Fragment Containing Genomic Murine FGFRβ

As noted above, the two splice variants muFGFR-β and FGFR-γ do not contain the classical receptor tyrosine kinase domain present in other known FGF receptors. In order to investigate whether FGFR5 contains a splice variant with a classical receptor tyrosine kinase (RTK) domain, the genomic DNA of FGFR5 was sequenced as follows.

Mouse genomic DNA was isolated from L929 cells using standard techniques. A genomic polynucleotide fragment containing murine FGFRβ was PCR amplified using primers MS157 and MS166 (SEQ ID NO: 11 and 12, respectively). The 1.4 kb polynucleotide fragment was cloned into a T-tailed pBluescript SK²⁺ vector. The sequence of the insert of this plasmid was determined using standard primer walking sequencing techniques. The sequence of the genomic fragment containing murine FGFRβ is given in SEQ ID NO: 9. This sequence extends from the 3′ untranslated region to the end of the mature FGFR5 receptor minus the signal sequence. No alternative exons containing an RTK domain were identified.

### Example 7

### Stimulation of Cell Growth by Murine FGFR5β and FGFR5γ

The stimulation of RAW264.10 cells (Hamilton et al., J. Exp. Med. 148:811-816, 1978) and peripheral blood mononuclear cells (PBMC) in the presence of the murine FGFRβ and FGFRγ (also referred to herein as FGFR5β and FGFR5γ, respectively) was demonstrated as follows. RAW264.10 cells are derived from a murine macrophage cell line generated from BALB/c mice, and are macrophage and osteoblast precursors.

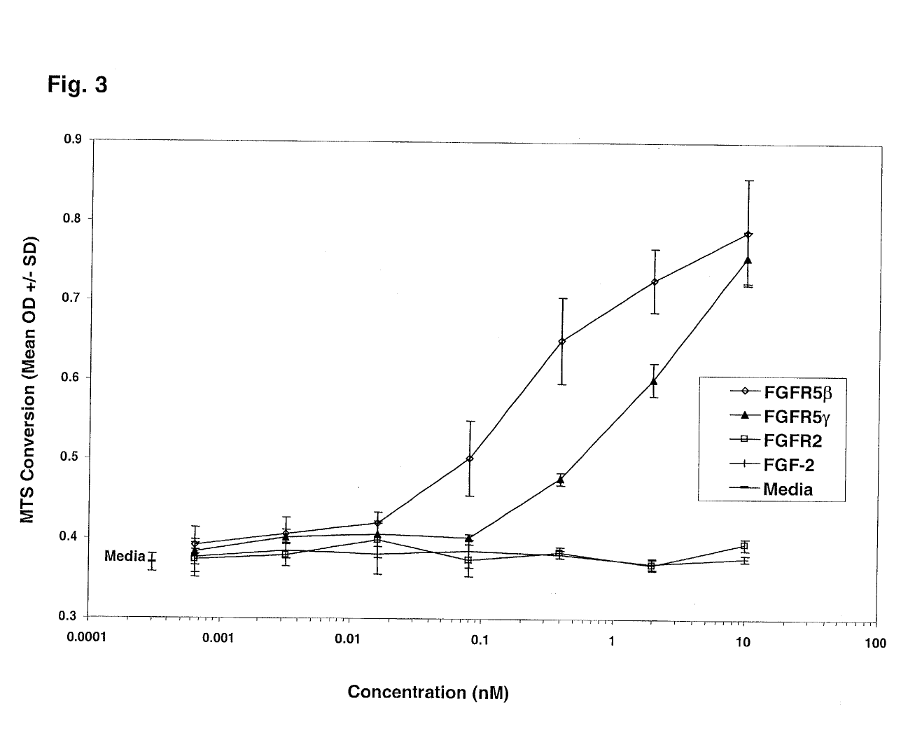

The murine FGF receptor homolog, muFGFRβ, and splice variant FGFRγ (SEQ ID NO: 2 and 3, respectively) were expressed in mammalian cells and purified as murine FGFR5β Fc fusion protein and FGFR5γ Fc fusion protein as described above. The FGFR5β and FGFR5 γ Fc fusion proteins were titrated from 10 nM in 0.05 ml media (DMEM supplemented with 5% FBS, 2 mM L-glutamine (Sigma, St Louis Mo.), 1 mM sodium pyruvate (Life Technologies, Gibco BRL, Gaithersburg Md.), 0.77 mM L-asparagine (Sigma), 0.2 mM arginine (Sigma), 160 mM penicillin G (Sigma), 70 mM dihydrostreptomycin sulfate (Boehringer Marnheim, Roche Molecular Biochemicals, Basel, Switzerland) in a 96 well flat-bottomed microtitre plate. Purified human FGFR2 Fc fusion protein was used as control and titrated from 10 nM. RAW264.10 cells were added to each well in 0.05 ml media at a concentration of 2×10⁴ cells/ml. The plate was incubated at 37° C. in a humidified atmosphere containing 10% CO₂ for 4 days. Cell growth was determined by MTS dye conversion and quantified using an ELISA reader. As shown in FIG. 3, both murine FGFR5β and FGFR5γ Fc fusion proteins stimulated the growth of RAW264.10 cells at concentrations of 100 pM and greater.

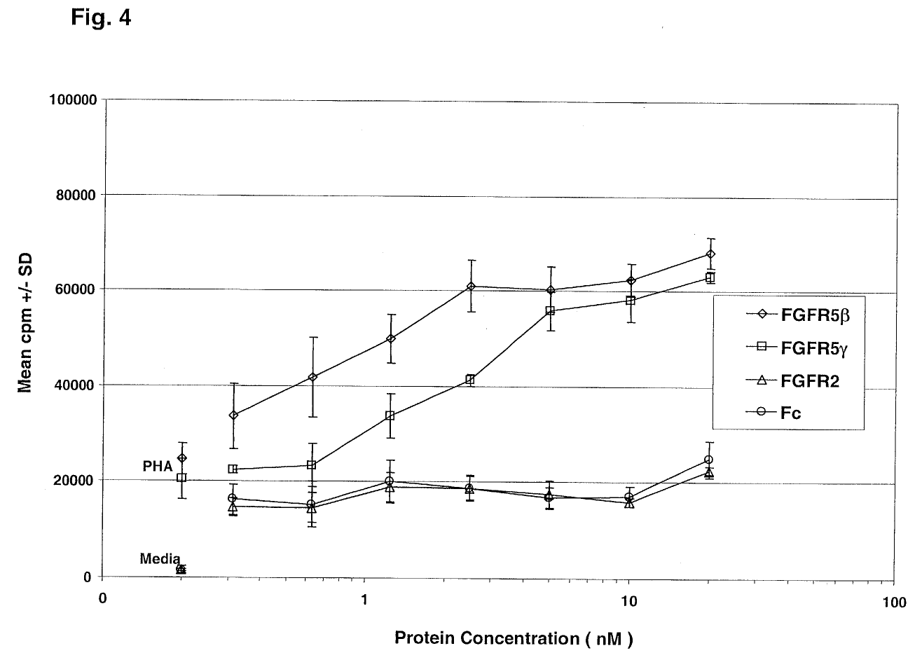

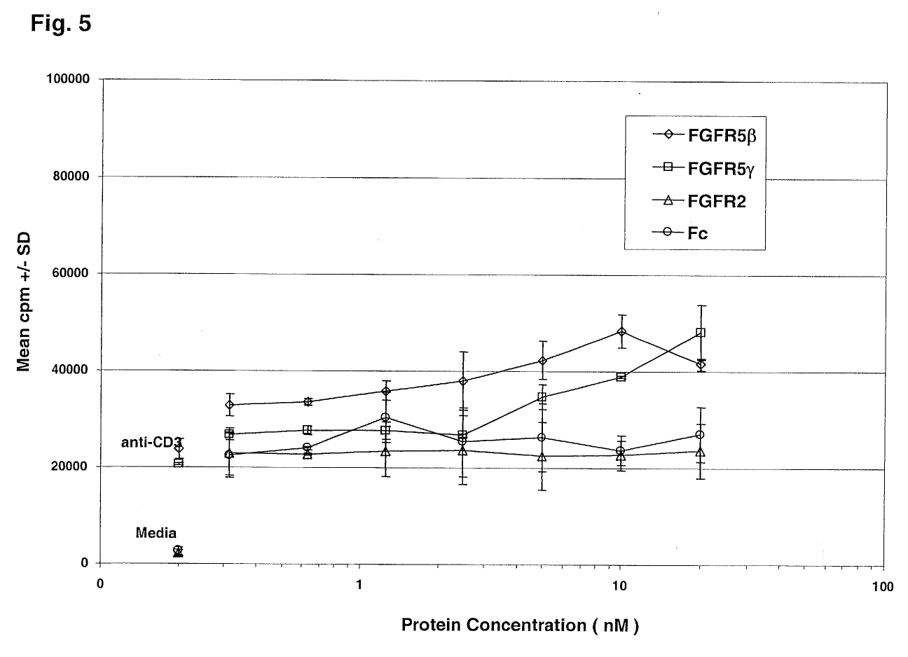

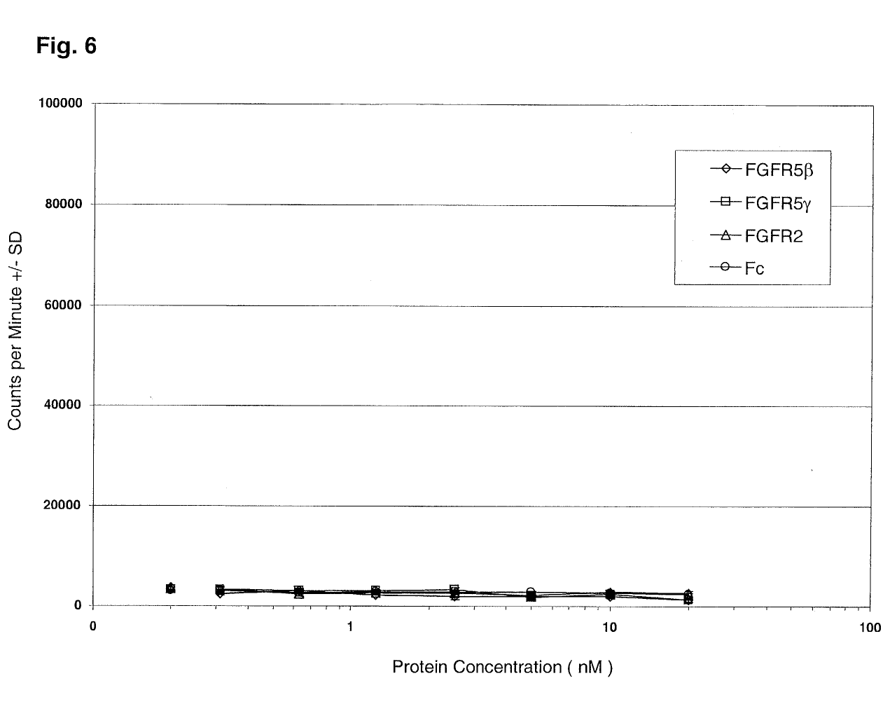

Purified FGFR5β and FGFR5γ Fc fusion proteins were titrated from 20 nM into 0.1 ml media per well of 96 well microtiter plates. Purified human FGFR2 Fc fusion protein and human IgG Fc were used as controls. PBMC were harvested from blood by density gradient centrifugation and resuspended in media to a concentration of 2×10⁶ cells/ml. Phytokemagglutinin (PHA), Pokeweed mitogen (PWM), anti-CD3 antibody or media was added to the PBMC and 0.1 ml of cells dispensed to each well. The plates were incubated for 3 days at 37° C. in a humidified atmosphere containing 5% CO₂ in air. Cell proliferation was quantified by pulsing the plates with tritiated thymidine for the final 16 hours of culture. The cells were then harvested and tritiated thymidine incorporation enumerated by standard liquid scintillation counting. FIGS. 4-6 show that murine FGFR5β and FGFR5γ fusion proteins enhanced the proliferation of PBMC activated with either PHA or anti-CD3 but did not induce the proliferation of PBMC on their own. Stimulation of proliferation was not observed with human FGFR2 Fc fusion protein or human IgG Fc.

These results demonstrate that FGFR5β and FGFR5γ are immunostimulatory molecules that directly activate a macrophage cell line. The macrophage cell line used in these assays (RAW264. 10) has previously been shown to differentiate into osteoblasts when stimulated with a variety of known bone morphogenic agents. The effects of FGFR5β and FGFR5γ on these cells suggest that these molecules may also stimulate the differentiation and activation of osteoblasts. Weidemann and Trueb (Genomics 69:275-279, 2000), have shown that FGFR5 is expressed in cartilaginous tissues. When combined with the data provided above, this suggests that FGFR5 may play a role in bone formation and may therefore have applications in fracture repair and bone diseases, such as osteoporosis and osteopetrosis.

### EXAMPLE 8

### Stimulation of Proliferation of Adherent Peripheral Blood Mononuclear Cells (PBMC) By Murine FGFR5β and FGFR5γ

Stimulation of PBMC to adhere to plastic by murine FGFR5β and FGFR5γ Fc fusion proteins was demonstrated as follows.

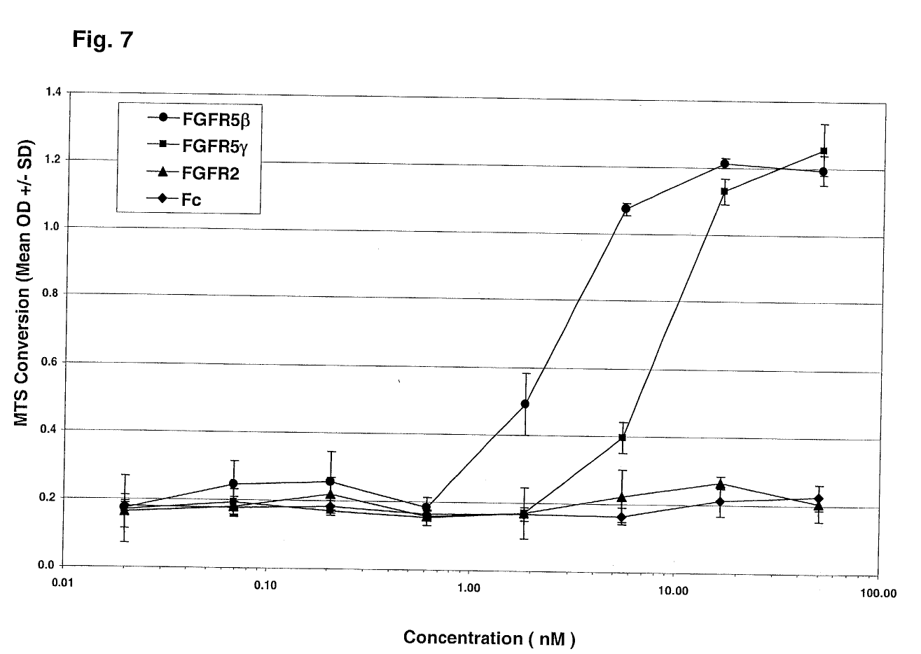

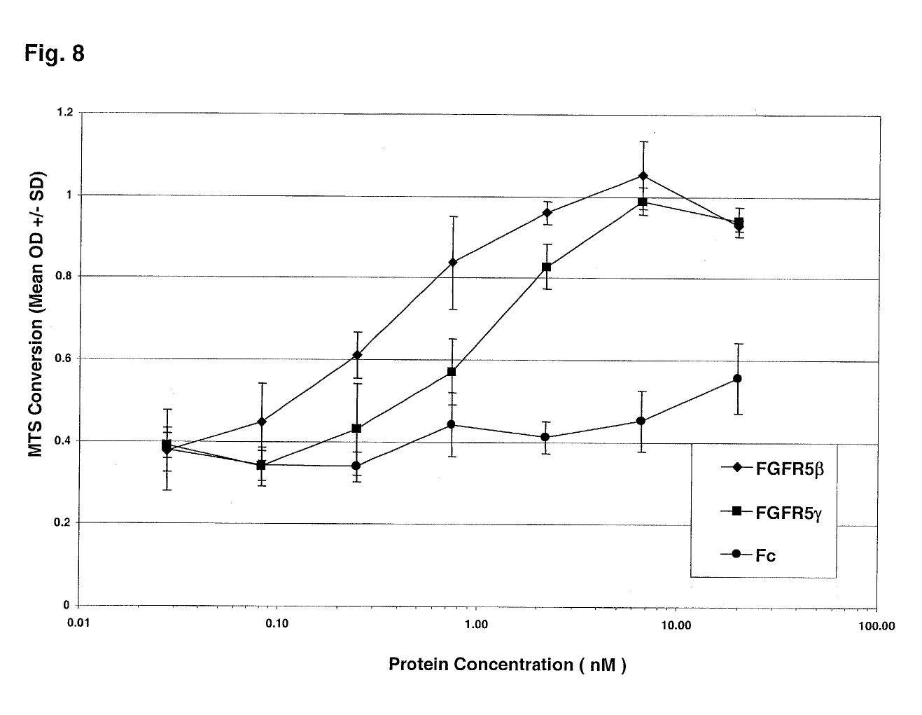

MuFGFR5β and muFGFR5γ (SEQ ID NO: 2 and 3, respectively) were expressed in mammalian cells and purified as Fc fusion proteins as described above. The muFGFR5β and muFGFR5γ Fc fusion proteins were titrated from 10 nM into 0.1 ml media per well of 96 well microtitre plates. Peripheral blood mononuclear cells (PBMC) were harvested from blood by density gradient centrifugation and resuspended in media to a concentration of 2×10⁶ cells/ml. PHA or media (RPMI 1640 supplemented with 5% FBS, 2 mM L-glutamine (Sigma), 160 mM penicillin G (Sigma), and 70 mM dihydrostreptomycin sulfate (Boehringer Mannheim) was added to the PBMC and 0.1 ml of cells dispensed to each well. The plates were incubated for 3 days at 37° C. in a humidified atmosphere containing 5% CO₂ in air. The non-adherent cells were removed with three media washes. Media (0.05 ml) containing MTS/PES solution (CellTiter96 Aqueous One Solution Cell Proliferation Assay, Promega, Madison, Wis.) was dispensed to each well and the plate incubated for 4 hrs before the degree of dye conversion was quantified using a 96 well ELISA reader. FIGS. 7 and 8 show that muFGFR5β and muFGFR5γ Fc fusion proteins stimulated the adherence and proliferation of adherent PBMC in a dose dependent manner and that PHA stimulation augmented this effect. These results demonstrate that FGFR5β and FGFR5γ are able to enhance the proliferative effects of known immunostimulatory molecules on a mixed population of human haemopoietic cells, namely PBMC.

### EXAMPLE 9

Activation of Natural Killer Cells by Murine FGFR5β and FGFR5γ

Activation of Natural Killer (NK) cells by muFGFR5β and muFGFR5γ Fc fusion proteins was demonstrated as follows.

Peripheral blood mononuclear cells (PBMC) were harvested from blood by density gradient centrifugation and resuspended in media (RPMI 1640 supplemented with 5% FBS, 2 mM L-glutamine (Sigma), 160 mM penicillin G (Sigma), 70 mM dihydrostreptomycin sulfate (Boehringer Mannheim)) to a concentration of 2×10⁶ cells/ml. Purified muFGFR5β and muFGFR5γ Fc fusion protein were added to the cells at a concentration of 10 nM and the cells were cultured in 6 well plates (3 ml/well) for 3 days at 37° C. in a humidified atmosphere containing 5% CO₂ in air. Purified human FGFR2 Fc fusion protein was used as control. The non-adherent cells were removed with three media washes. The adherent cells were collected by light trypsinization and scraping. The cells were washed into staining buffer and their phenotype determined by standard flow cytometric techniques using NK cell marker CD56 and a control isotype antibody.

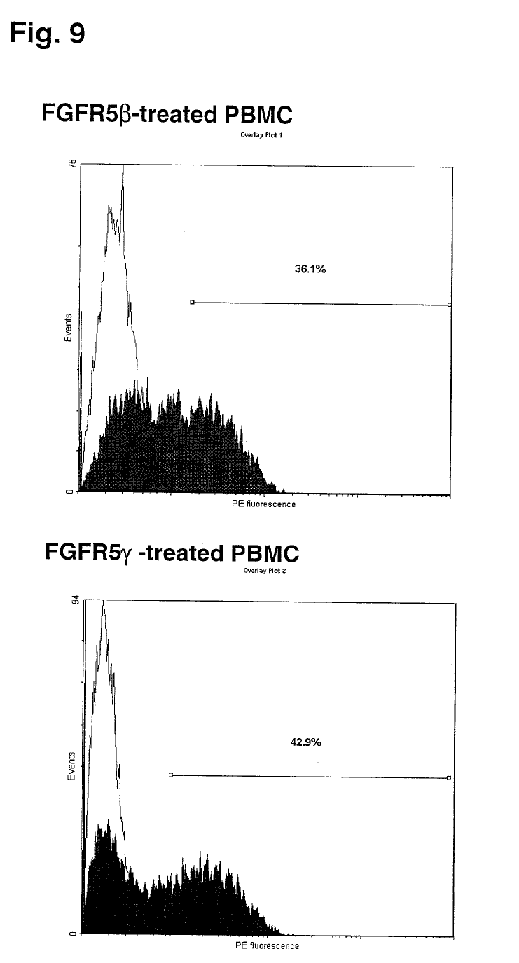

As shown in FIG. 9, muFGFR5β and muFGFR5γ Fc fusion proteins stimulated the adherence and/or growth of adherent cells from human PBMC, with approximately 50% of these cells being NK cells. The filled histograms represent the adherent PBMC stained with the NK cell marker CD56 and the open histograms represent the same cells stained with the isotype-matched control antibody. FGFR2 did not stimulate the adherence of PBMC and therefore there were no cells to analyze from these cultures. These results demonstrate that FGFR5β and FGFR5γ are immunostimulatory molecules that directly activate NK cells. These results, plus those provided in Example 8, above, show that FGFR5 can enhance immune responses, and may thus be usefully employed to enhance vaccine responses and anti-cancer therapies.

### EXAMPLE 10

### Immunoprecipitation of a 20-30 KDA Surface Protein from RAW264.10 Cells by MuFGFR5β and MuFGFR5γ FC Fusion Proteins

Immunoprecipitation of a 20-30 kDa protein from the surface of RAW264. 10 cells by murine FGFR5β and FGFR5γ Fc fusion proteins, but not FGFR2 or control (murine MALA13003) Fc fusion proteins was demonstrated as follows. Murine MALA13003 is a novel receptor of unknown function.

Preparation of Protein a Beads Bound with Murine FGFRβ and FGFR5γ Fc Fusion Proteins

Two μl Protein A Beads (Protein A Ceramic HyperDF, Gibco BRL) were dispensed into 4 microfuge tubes. The beads were washed three times in IP wash buffer (50 mM Tris.HCl pH 7.5, 0.5 M NaCl, 1 mM CaCl₂, 1 mM MgCl₂, 0.1% Tween-20). The IP wash buffer was removed and 20 μg purified murine FGFR5β, FGFR5γ, FGFR2 or murine MALA 31003 Fc fusion proteins was added to each microfuge tube and incubated on a rocking platform for either 2-3 hours at room temperature or overnight at 4° C. The beads were washed three times with IP wash buffer and centrifuged at 3,000 rpm for 2 min.

Preparation of Cells

RAW264.10 cells were harvested when at 75% confluence by using single strength trypsin and washed in PBS. After counting, the cell concentration was adjusted to 5×10⁶ cells/ml in 5 ml PBS.

Biotinylation

A stock solution of N-hydroxysuccinimido-biotin (NHS-Biotin, Sigma) at 10 mg/ml was prepared in PBS. An aliquot of this stock biotin solution was added immediately to the RAW264.10 cell suspension to a final concentration of 100 μg/ml biotin and mixed carefully. The cell suspension was incubated at room temperature for 60 min on a rocking platform to prevent the cells from settling. The cells were washed three times by centrifugation at 1,000 rpm for 7 min and resuspension in glycine buffer (PBS, 10 mM glycine, pH 7.5) to block all free NHS-Biotin sites. The glycine was removed by washing the cells twice in PBS. The cells were aliquoted into four 1 ml portions of 5×10⁶ cells/ml each and pelleted by centrifugation.

Extraction of Soluble Proteins

One ml of extraction buffer (50 mM Tris.HCl pH 7.5, 0.15 M NaCl, 1 mM CaCl₂, 1 mM MgCl₂, 1% NP40) was added to each cell pellet. Cells were disrupted by using a syringe until the cell pellet was solubilized. The solubilized cells were centrifuged at 10,000 rpm for 10 min at 4° C. and the supernatant was collected.

Immunoprecipitation

The cell lysates were pre-cleared twice by adding 2 μl washed Protein A beads and incubating for 1 hour at room temperature. The cell suspension was centrifuged and the supernatant collected. One ml pre-cleared lysate was added to each of the microfuge tubes containing 2 μl protein A beads coated with FGFR5β, FGFR5γ, FGFR2 or control Fc fusion proteins and incubated overnight at 4° C. on a rocking platform. The beads were washed four times in IP wash buffer with a centrifugation step (4,000 rpm for 2 min). After the first wash, the bead suspension was transferred to a new tube so that the final sample was not contaminated with non-bound recombinant protein that may be stuck to the side of the tube. The supernatant was aspirated leaving approximately 20 μl of liquid on the beads. After the final wash, all the remaining 20 μl of supernatant on the beads was removed and the beads resuspended in 20 μl RSB (0.08 M Tris.HCl pH 6.8, 0.01 M DTT (Dithiothreitol), 2% SDS, 10% glycerol).

Polyacrylamide Gel Electrophoresis

The prepared beads were run on two resolving polyacrylamide gels following standard procedures. One gel was stained with Coomassie Blue and the second gel was blotted onto PVDF (polyvinylidene difluoride) membrane (Immobilon-P Transfer Membrane, Millipore, Bedford Mass.) by Western transfer following standard procedures. The blot was developed using ECL Western Blotting Detection Reagent (Amersham Pharmacia, Uppsala, Sweden) and exposed to Scientific Imaging film (Agfa Curix Blue HC-S Plus).

A protein of 20 to 30 kDa molecular weight was immunoprecipitated from the surface of RAW264.10 cells by the murine FGFR5β and FGFR5γ Fc fusion proteins, but not FGFR2 or control Fc fusion proteins. This precipitated protein is likely to be the ligand, or a component of the ligand complex, recognized by FGFR5. Studies have indicated that FGF receptors can use other molecules in addition to fibroblast growth factors as ligands and that N-CAM and other molecules can trigger FGFR signaling (Alitalo, Nature Cell Biol. 3:E157-E159, 2001; and Cavallaro et al. Nature Cell Biol. 3:650-657, 2001).

### EXAMPLE 11

### Stimulation of Gene Expression in Human Monocytes by Murine FGFR5β Fc Fusion Protein

The ability of FGFR5 to stimulate gene expression in human monocytes was examined as follows.

Monocytes were purified from human peripheral blood mononuclear cells by adherence for 2 hours at 37° C. Cells were stimulated with 100 nM of soluble FGFR5β human IgG Fc fusion protein or soluble FGFR2 human IgG Fc fusion protein. After 0 and 12 hours the adherent monocytes were collected and total RNA extracted from the cells using Trizol reagent (Invitrogen Corp., Carlsbad Calif.) following the manufacturer's instructions. The RNA was amplified and aminoallyl UTP incorporated using the Ambion MessageAmp aRNA kit (Ambion Inc, Austin Tex.) following the manufacturer's instructions. The extracted amplified RNA from the FGFR5β and FGFR2-treated cells were labeled with either Cy3 or Cy5 dye (Amersham Pharmacia Biotech, Buckinghamshire UK), respectively, by indirect aminoallyl dUTP labeling and hybridized to 2 Clontech Atlas Glass 3.8 gene microarrays (BD Biosciences Clontech, Palo Alto, Calif.). The slides were washed, scanned and analyzed using Axon GenePix scanner and software (Axon Instruments Inc., Union City, Calif.). Where indicated, quantitative PCR was used to validate the microarray data and quantify the mRNA for genes not present on the array. Primers and probe sets were purchased from Perkin Elmer/Applied Biosystems (Foster City, Calif.) and MWB Biotech (Ebersberg, Germany) and all PCR reactions were run on a Perkin Elmer/Applied Biosystems 7700 following the manufacturer's instructions. Treatment of monocytes with FGFR5β up-regulated the expression of the 26 genes listed in Table 1 below. The up-regulation of three of the genes was confirmed by quantitative PCR. In addition, the expression of eight human cytokines was analyzed by quantitative PCR and the results of this analysis are shown in Table 1. FGFR5 stimulated a dramatic up-regulation in the levels of osteopontin (OPN) and TGFP but had only modest effects on the other cytokines. This profile of gene expression is very unlike that described for other stimulators of monocytes such as LPS, Mycobacterium tuberculosis, GM-CSF and M-CSF, which stimulate modest OPN expression but pronounced expression of pro-inflammatory cytokines such as IL-1β, IL-6, IL-8 IL-10, IL-12 and TNFα (Rosenberger et al., J. Immunol. 164:5894-904, 2000, Suzuki et al., Blood 96:2584-2591, 2000; Hashimoto et al., Blood 94:837-844, 1999; Hashimoto et al., Blood 94:845-852, 1999; Boldrick et al., Proc. Natl. Acad. Sci. USA 99:972-977, 2002; Ragno et al., Immunol. 104:99-108, 2001).

TABLE 1
Genes up-regulated in monocytes following treatment with FGFR5

| | | Microarray | Quantitative |
|---|---|---|---|
| | | Fold up- | PCR Fold |
| | GENBANK | regulation | up-regulation |
| Secreted Molecules | | | |
| Osteopontin | NM_000582 | 4.95 | 48.4 |
| Interferon, alpha 8 | NM_002170 | 2.27 | ND |
| EXODUS | NM_004591 | 2.27 | 6.3 |
| IL-1β | XO2532 | Not Determined | 3.4 |
| | | (ND) | |
| IL8 | NM_000584 | ND | 5.5 |
| IL-10 | NM_000572 | ND | undetectable |
| IL-12 p35 | NM_000882 | ND | undetectable |
| IL-12p40 | NM_002187 | ND | undetectable |
| IL-20 | NM_018724 | ND | undetectable |
| TGFβ | NM_000660 | ND | 27.3 |
| TNFα | XO1394 | ND | 4.0 |
| Channels and Receptors | | | |
| MICA | NM_000247 | 2.08 | 4.7 |
| TIE1 | NM_005424 | 3.30 | ND |
| Calcium channel, | NM_000726 | 2.44 | ND |
| voltage-dependent, beta 4 | | | |
| subunit | | | |
| LDL receptor-related | NM_004631 | 2.20 | ND |
| protein 8 | | | |
| Cytoskeletal Molecules | | | |
| Myosin VI | NM_004999 | 1.89 | ND |
| Myosin, heavy | NM_005963 | 2.12 | ND |
| polypeptide 1 | | | |
| Troponin C, slow | NM_003280 | 1.88 | ND |
| Kinectin 1 kinesin | NM_004986 | 1.73 | ND |
| receptor | | | |
| Signalling Molecules | | | |
| Protein kinase C, iota | NM_002740 | 2.26 | ND |
| Protein tyrosine | NM_002833 | 1.85 | ND |
| phosphatase, non- | | | |
| receptor type 9 MEG-2 | | | |
| Importin alpha 6 | NM_002269 | 2.17 | ND |
| Protein kinase, X-linked | NM_005044 | 1.92 | ND |
| Suppression of | NM_005418 | 3.16 | ND |
| tumorigenicity 5 | | | |
| RAR-related orphan | NM_006914 | 2.08 | ND |
| receptor B | | | |
| Zinc finger protein 124 | NM_003431 | 2.94 | ND |
| HZF-16 | | | |
| Metabolism | | | |
| Ubiquitin-conjugating | NM_003341 | 2.41 | ND |
| enzyme | | | |
| Transplantation antigen | NM_003313 | 2.48 | ND |
| P35B | | | |
| UDP glycosyltransferase | NM_001075 | 2.35 | ND |
| 2 | | | |
| Alcohol dehydrogenase 2 | NM_000668 | 2.41 | ND |
| Solute carrier family 18 | NM_003053 | 2.07 | ND |
| vesicular monoamine, | | | |
| member 1 | | | |
| Seryl-tRNA synthetase | NM_006513 | 1.88 | ND |
| Other | | | |
| H1 histone family, | NM_005325 | 1.99 | ND |
| member 1 | | | |
| Chr. 8 open reading | NM_004337 | 2.08 | ND |
| frame 1 | | | |

These results demonstrate that FGFR5 is a potent, and almost specific, stimulator of osteopontin expression. Osteopontin (OPN) is a multifunction protein secreted by activated macrophage that shares most of the functions described for FGFR5.

More specifically, OPN is a potent immunostimulatory molecule (O'Regan et al., Immunol. Today 21:475-478, 2000) that stimulates macrophage adherence, activation, cytokine secretion and growth. It has been shown to be a regulator of T cell responses in that it augments CD3-induced proliferation, IFNγ production and CD40 ligand expression, and enhances Th1 and inhibits Th2 cytokine expression. It directly induces macrophage to produce IL-12 and inhibits IL-10 expression by LPS stimulated macrophages (Ashkar et al., Science 287:860-864, 2000). OPN has also been shown to induce B cell proliferation and auto-reactive antibody production. It appears that OPN may preferentially activate a CD5+subset of B cells and induce the production of auto-antibodies.

Osteopontin has been linked with a number of pathophysiological states including a variety of tumors; autoimmune diseases such as multiple sclerosis (MS), systemic lupus erythematosus (SLE), diabetes and rheumatoid arthritis; granulomatous inflammation such as sarcoidosis and tuberculosis; and pathological calcifications such as kidney stones and atherosclerosis (Giachelli and Steitz, Matrix Biol. 19:615-622, 2000). Elevated levels of OPN are found in the sera of SLE patients and the autoimmune-prone MRL mice. Recently two groups described a central role for OPN in multiple sclerosis (Chabas et al., Science 294:1731-1735, 2001; Jansson, J. Immunol. 168:2096-2099, 2002). OPN is prevalent in the plaques of MS patients and, due to its immunostimulatory properties, it was proposed that OPN plays a role in the progression of the disease. This effect was demonstrated in experimental allergic encephalopathy (EAE), the murine model for MS. Mice that lacked the OPN gene were resistant to progressive EAE and had frequent remissions when compared to wild type mice expressing OPN.

SLE is an autoimmune disorder that effects 24 out of 100,000 individuals in the USA. Afflicted individuals usually develop nephritis, arthritis and dermatitis. Auto-antibody production, complement activation, immune complex deposition, Fc receptor ligation and leukocyte infiltration of the target organs are among the immunopathogenic events. The chromosomal location of FGFR5 is 4p16. Genetic screens on large numbers of SLE patients show that a mutation at this location is associated with disease. FGFR5 sequence analysis may thus be used to identify individuals at risk for SLE by determining whether a mutation exists.

OPN has also been shown to function in bone remodelling by inhibiting calcification. Inhibition of OPN expression, by reducing the level or binding of FGFR5, may thus be useful in the treatment of osteoporosis.

Many of the effects described for FGFR5 may be mediated by its ability to induce high levels of osteopontin expression. Osteopontin is clearly a key molecule in the progression of a number of disease processes and therefore regulators of osteopontin expression, such as FGFR5, are targets for therapeutics for osteopontin-mediated diseases, including SLE, vasculitis, atherosclerosis, nephritis and arthritis.

### EXAMPLE 12

### Analysis of FGFR5 Expression Using FGFR5-Specific Antibodies

Polyclonal antibodies were generated to the extracellular domain of FGFR5β by immunizing rabbits with murine FGFR5β extracellular domain fused to human IgG1 Fc fragment emulsified in complete Freund's adjuvant. The FGFR5-specific immune response was boosted by three subcutaneous injections at three weekly intervals with the same protein and then twice with pure murine FGFR5β extracellular domain protein. Antisera were collected from the rabbits and the IgG purified by Protein A affinity chromatography. Antibodies raised to the human IgG Fc portion of the immunogen were removed by absorption to Sephadex beads coated with human IgG. The resultant polyclonal antibody specifically reacted with human and mouse FGFR5 but did not recognize human FGFR1, 2, 3, or 4 Fc fusion proteins (purchased from R&D Systems, Minneapolis Minn.) in ELISA or by Western blotting.

Immunohistochemical analysis of human normal and diseased tissue arrays (SuperBioChips Laboratories, Seoul, Korea) revealed that FGFR5 is expressed in a minor population of cells in the red pulp region of the spleen. These cells are likely to be macrophages. FGFR5-expressing macrophage-like cells were also found in a number of tissues including the stomach, lung and small intestine. FGFR5 expression was also detected in skeletal muscle, skin and kidney. In addition, expression of FGFR5 was found in tissue biopsies from a hepatocellular carcinoma and a squamous cell carcinoma.

FGFR5 is expressed in cells within the islets of Langerhans of the pancreas and may therefore play a role in diabetes (see, Kim et al. Biochim. Biophys. Acta 1518:152-156, 2001), especially given the immunostimulatory properties of this molecule. Recent studies have shown that FGF-like molecules are involved in cardiac disease (Liao Nature Medicine 8:20-21, 2002; and Asakura et al. Nature Medicine 8:35-40, 2002) indicating a possible role for FGFR5 in such disorders.

### EXAMPLE 13

### Effects of FGFR5 Administration in vivo

The effects of administration of FGFR5β protein in vivo were examined in three experiments as follows.

Experiments 1 and 2

Experiment 1 used BALB/cByJ mice and experiment 2 used C3H/HeJ mice. Both sets of mice were injected subcutaneously with 5 ug (55 nM in 0.1 ml PBS) of murine FGFR5β extracellular domain (ECD)—murine IgG3 Fc fusion protein in the morning and the same dose in the evening (i.e. each mouse received 10 ug per day) for five days. Control mice received PBS alone. On the sixth day, the mice were sacrificed and the draining lymph nodes (axillary and lateral axillary) were removed. A single cell suspension was generated form the lymph nodes of each mouse and the number of cells collected from each mouse was determine by trypan blue viability counting using a haemocytometer. The lymph node cells collected from the FGFR5-treated mice were then pooled. The lymph node cells collected form the PBS-treated mice were amalgamated into a separate pool of cells. The cells from both the FGFR5 and PBS-treated mice were then stained for the cell surface antigens listed in Table 2, below, and analysed by flow cytometry.

Experiment 3

In this experiment, C3H/HeJ mice were injected subcutaneously with 10 ug (110 nM in 0.1 ml PBS) of murine FGFR5β ECD—human IgG1 Fc fusion protein in one injection per day for 5 days. While the treatment regime differed from that used in Experiments 1 and 2 above, the total dose of protein administered to the mice did not alter. Control mice were administered human IgG1 Fc fragments alone. On the sixth day, the mice were sacrificed and the draining lymph nodes (axillary and lateral axillary) removed. The number of cells collected from each mouse and the presence of cell surface antigens was determined as described above.

As shown in Table 2, in vivo administration of FGFR5 was found to stimulate lymphadenopathy, or enlargement of the lymph nodes. When compared to mice treated with Fc protein, the frequency of B cells doubled in the draining lymph nodes of FGFR5-treated mice. An analysis of the cell cycle state of the B cells by flow cytometry indicated that they were not expanding but were either selectively migrating or being retained in the lymph nodes. This is consistent with the data provided above showing that FGFR5 causes the growth of macrophage but not T or B cells in culture. The cells are, however, activated as there is an increase in the number of cells expressing the very early activation antigen, CD69.

TABLE 2
Comparison of 3 in vivo experiments testing the effects of
soluble FGFR5 in mice

| | | Experiment 1 | | Experiment 2 | | Experiment 3 | |
|---|---|---|---|---|---|---|---|
| | | Balb/c | | C3H/HeJ | | C3H/HeJ | |
| | | Murine | | Murine | | Human | |
| | Cell type | Fc | | Fc | | Fc | Human |
| Markers | recognized | FGFR5 | PBS | FGFR5 | PBS | FGFR5 | Fc |
| CD3 | T cell | 63 | 81 | 59 | 82 | 32 | 67 |
| CD19 | B cell | 35 | 21 | 39 | 16 | 61 | 26 |
| Class II | B cell and | 41 | 20 | ND* | ND | ND | ND |
| | macro- | | | | | | |
| | phage | | | | | | |
| CD45R | B cell | ND | ND | ND | ND | 72 | 31 |
| CD69 | Activated | 23 | 14 | 18 | 10 | 21 | 10 |
| | cells | | | | | | |

*ND = Not determined

SEQ ID NOS: 1-14 are set out in the attached Sequence Listing. The codes for polynucleotide and polypeptide sequences used in the attached Sequence Listing conform to WIPO Standard ST.25 (1988), Appendix 2.

All references cited herein, including patent references and non-patent references, are hereby incorporated by reference in their entireties.

Although the present invention has been described in terms of specific embodiments, changes and modifications can be carried out without departing from the scope of the invention which is intended to be limited only by the scope of the appended claims.

We claim:

1. An isolated polypeptide comprising a sequence selected from the group consisting of: SEQ ID NO: 5-8 and 13-15.

2. An isolated polypeptide comprising a sequence selected from the group consisting of:
(a) sequences having at least 75% identity to a sequence provided in SEQ ID NO: 5-8 and 13-15;
(b) sequences having at least 90% identity to a sequence provided in SEQ ID NO: 5-8 and 13-15; and
(c) sequences having at least 95% identity to a sequence provided in SEQ ID NO: 5-8 and 13-15,
wherein the polypeptide possesses at least one functional property that is substantially the same as a functional property of a sequence of SEQ ID NO: 5-8 and 13-15.

3. An isolated polynucleotide that encodes a polypeptide according to any one of claims 1 and 2.

4. An isolated polynucleotide of claim 3, wherein the polynucleotide comprises a sequence selected from the group consisting of: sequences provided in SEQ ID NO: 1-4 and 9.

5. An isolated polynucleotide comprising a sequence selected from the group consisting of:
(a) complements of a sequence provided in SEQ ID NO: 1-4 and 9;
(b) reverse complements of a sequence provided in SEQ ID NO: 1-4 and 9;
(c) reverse sequences of a sequence provided in SEQ ID NO: 1-4 and 9;
(d) sequences having at least 75% identity to a sequence provided in SEQ ID NO: 1-4 and 9; and
(e) sequences having at least 90% identity to a sequence provided in SEQ ID NO: 1-4 and 9; and
(f) sequences having at least 95% identity to a sequence of SEQ ID NO: 1-4 and 9.

6. An isolated polynucleotide comprising a sequence selected from the group consisting of: (a) sequences that are a 200-mer of an isolated polynucleotide according to any one of claims 3, 4 and 5; (b) sequences that are a 100-mer of an isolated polynucleotide according to any one of claims 3, 4 and 5; and (c) sequences that are a 40-mer of an isolated polynucleotide according to any one of claims 3, 4 and 5.

7. An expression vector comprising an isolated polynucleotide according to any one of claims 3-6.

8. A host cell transformed with an expression vector according to claim 7.

9. An isolated polypeptide comprising at least a functional portion of an amino acid sequence selected from the group consisting of sequences provided in SEQ ID NO: 5-8 and 13-15.

10. A fusion protein comprising at least one polypeptide according to any one of claims 1, 2 and 9.

11. A composition comprising an isolated polypeptide according to any one of claims 1, 2 and 9 and at least one component selected from the group consisting of: physiologically acceptable carriers and immunostimulants.

12. A composition comprising an isolated polynucleotide according to any one of claims 3-6 and at least one component selected from the group consisting of: physiologically acceptable carriers and immunostimulants.

13. A composition comprising a fusion protein according to claim 10 and at least one component selected from the group consisting of: physiologically acceptable carriers and immunostimulants.

14. A method for the treatment of a disorder of the immune system in patient, comprising administering to the patient a composition according to any one of claims 11-13.

15. A method for the treatment of cancer in a patient, comprising administering to the patient a composition according to any one of claims 11-13, wherein the cancer is selected from the group consisting of epithelial, lymphoid, myeloid, stromal and neuronal cancers.

16. A method for the treatment of a viral disorder in a patient, comprising administering to the patient a composition according to any one of claims 11-13.

17. The method of claim 16, wherein the viral disorder is HIV-infection.

18. A method for the treatment of a fibroblast growth factor-mediated disorder in a patient, comprising administering a composition according to any one of claims 11-13.

19. A method for modulating an immune response in a patient, comprising administering to the patient a composition according to any one of claims 11-13.

20. A method for inhibiting the expression of osteopontin in a population of cells, comprising reducing the amount of a polypeptide in the cells, the polypeptide comprising an amino acid sequence selected from the group consisting of:
(a) a sequence provided in SEQ ID NO: 5-8 and 13-15;
(b) sequences having at least 75% identity to a sequence provided in SEQ ID NO: 5-8 and 13-15
(c) sequences having at least 90% identity to a sequence provided in SEQ ID NO: 5-8 and 13-15; and
(d) sequences having at least 95% identity to a sequence provided in SEQ ID NO: 5-8 and 13-15.

21. A method for inhibiting the expression of osteopontin in a population of cells, comprising inhibiting the activity of a polypeptide in the cells, the polypeptide comprising an amino acid sequence selected from the group consisting of:
(a) a sequence provided in SEQ ID NO: 5-8 and 13-15;
(b) sequences having at least 75% identity to a sequence provided in SEQ ID NO: 5-8 and 13-15;
(c) sequences having at least 90% identity to a sequence provided in SEQ ID NO: 5-8 and 13-15; and
(d) sequences having at least 95% identity to a sequence provided in SEQ ID NO: 5-8 and 13-15.

22. The method of claim 21, wherein the method comprises contacting the cells with an antibody, or an antigen-binding fragment thereof that binds specifically to a polypeptide comprising an amino acid sequence selected from the group consisting of:
(a) sequences provided in SEQ ID NO: 5-8 and 13-15;
(b) sequences having at least 75% identity to a sequence provided in SEQ ID NO: 5-8 and 13-15;
(c) sequences having at least 90% identity to a sequence provided in SEQ ID NO: 5-8 and 13-15; and
(d) sequences having at least 95% identity to a sequence provided in SEQ ID NO: 5-8 and 13-15.

23. The method of claim 21, wherein the method comprises contacting the cells with an anti-sense oligonucleotide that binds specifically to a polynucleotide comprising a sequence selected from the group consisting of:
(a) sequences provided in SEQ ID NO: 1-4 and 9;
(b) sequences having at least 75% identity to a sequence provided in SEQ ID NO: 1-4 and 9;
(c) sequences having at least 90% identity to a sequence provided in SEQ ID NO: 1-4 and 9; and
(d) sequences having at least 95% identity to a sequence provided in SEQ ID NO: 1-4 and 9.

24. The method of claim 21, wherein the method comprises contacting the cells with a small interfering RNA molecule that corresponds to a polynucleotide comprising a sequence selected from the group consisting of:
(a) sequences provided in SEQ ID NO: 1-4 and 9;
(b) sequences having at least 75% identity to a sequence provided in SEQ ID NO: 1-4 and 9;
(c) sequences having at least 90% identity to a sequence provided in SEQ ID NO: 1-4 and 9; and
(d) sequences having at least 95% identity to a sequence provided in SEQ ID NO: 1-4 and 9.

25. A method for treating a disorder characterized by an elevated level of osteopontin, comprising administering an antibody, or an antigen-binding fragment thereof that binds specifically to a polypeptide comprising an amino acid sequence selected from the group consisting of:
(a) sequences provided in SEQ ID NO: 5-8 and 13-15;
(b) sequences having at least 75% identity to a sequence provided in SEQ ID NO: 5-8 and 13-15;
(c) sequences having at least 90% identity to a sequence provided in SEQ ID NO: 5-8 and 13-15; and
(d) sequences having at least 95% identity to a sequence provided in SEQ ID NO: 5-8 and 13-15.

26. A method for treating a disorder characterized by an elevated level of osteopontin, comprising administering an anti-sense oligonucleotide that binds specifically to a polynucleotide comprising a sequence selected from the group consisting of:
(a) sequences provided in SEQ ID NO: 1-4 and 9;
(b) sequences having at least 75% identity to a sequence provided in SEQ ID NO: 1-4 and 9;
(c) sequences having at least 90% identity to a sequence provided in SEQ ID NO: 1-4 and 9; and
(d) sequences having at least 95% identity to a sequence provided in SEQ ID NO: 1-4 and 9.

27. A method for treating a disorder characterized by an elevated level of osteopontin, comprising administering a small interfering RNA molecule that corresponds to a polynucleotide comprising a sequence selected from the group consisting of:
(a) sequences provided in SEQ ID NO: 1-4 and 9;
(b) sequences having at least 75% identity to a sequence provided in SEQ ID NO: 1-4 and 9;
(c) sequences having at least 90% identity to a sequence provided in SEQ ID NO: 1-4 and 9; and
(d) sequences having at least 95% identity to a sequence provided in SEQ ID NO: 1-4 and 9.

28. The method of any one of claims 25-27, wherein the disorder is selected from the group consisting of: multiple sclerosis; systemic lupus erythematosus; diabetes; rheumatoid arthritis; sarcoidosis; tuberculosis; kidney stones; atherosclerosis; vasculitis; nephritis; arthritis; and osteoporosis.
